(19) United States
(12) Patent Application Publication
(10) Pub. No.: US 2025/0364144 A1
(43) Pub. Date: Nov. 27, 2025

(54) CANCER PHARMACOPREVENTION THROUGH ARTIFICIAL INTELLIGENCE

(71) Applicant: THE BOARD OF TRUSTEES OF THE UNIVERSITY OF ILLINOIS, Urbana, IL (US)

(72) Inventors: Abdul ZAKKAR, Chicago, IL (US); Ameen SALAHUDEEN, Chicago, IL (US)

(21) Appl. No.: 19/218,073

(22) Filed: May 23, 2025

Related U.S. Application Data
(60) Provisional application No. 63/651,235, filed on May 23, 2024.

(51) Int. Cl. G16H 50/30; G06T 7/00; G06V 10/764; G06V 10/82; G16H 20/10; G16H 30/20

(52) U.S. Cl. CPC G16H 50/30; G06T 7/0012; G06V 10/764; G06V 10/82; G16H 20/10; G16H 30/20; G06T 2207/20084

(57) ABSTRACT

This invention relates to the use of artificial intelligence in methods of predicting and treating cancer with prophylactics/preventative agents. As compared to the current state of the art of clinical risk models or calculators for cancer risk and preventive measures, this invention integrates long-term risk via artificial intelligence inference of medical imaging cancer screens. The end result is less false-positives when one is predicted to be high risk for developing cancer, thus lowering the number needed to treat for a positive outcome of cancer prevention with a prophylactic.

### CROSS REFERENCE TO RELATED APPLICATIONS

The present application claims priority to U.S. Provisional Patent Application No. 63/651,235, filed May 23, 2024, the disclosure of which is incorporated by reference in its entirety.

### TECHNICAL FIELD

This invention relates to the use of artificial intelligence in methods of predicting and treating cancer with prophylactics/preventative agents. As compared to the current state of the art of clinical risk models or calculators for cancer risk and preventive measures, this invention integrates risk via artificial intelligence inference of medical imaging cancer screens. The end result is less false-positives when one is predicted to be high risk for developing cancer, thus lowering the number needed to treat for a positive outcome of cancer prevention with a prophylactic.

### BACKGROUND OF THE INVENTION

Cancer is the second leading cause of death in the United States (after heart disease) and of cancers, lung cancer leads followed by colorectal cancer, pancreatic cancer, and breast cancer. Cancer prevention has been proven effective with the use of vaccines in cervical cancer¹ as well as prophylactic or pharmacopreventive therapies such mastectomy in women with hereditary breast cancers or as anti-estrogen therapies in breast cancer^{2,3}. Pharmacoprevention, however, has had limited use in practice, owing to either lack of positive efficacy trials, or due to the limited risks and benefits as compared to adverse effects from preventive interventions. For example, the use of antiestrogens for primary breast cancer prevention has been controversial in part due to risk of thrombosis or uterine cancer from tamoxifen⁴. Another example, in lung cancer, the CARET study attempted to achieve pharmacoprevention using beta-carotene and retinol but did not demonstrate efficacy⁵. A commonality with these and other examples is the inability to determine cancer risk in a window of intervention when the benefit of the prophylactic (i.e. cancer risk reduction) outweighs the risk, cost, or side effects of the prophylactic measure. Achieving more precision and targeted efficacy with pharmacopreventive agents would improve the uptake and utilization of these agents and also lead to reduced cancer incidence and deaths.

Health disparities exist in cancer survival particularly in African American populations and women^{6,7}. Mortality reducing interventions such as lung cancer screening through Low Dose CT imaging LDCT)⁸ also have been shown to exhibit racial disparities. Specifically, LDCT screening eligibility criteria have been identified as too stringent in African Americans as measured by the eligibility to incidence ratio, where non-Hispanic Whites often have 100 eligible patients for every patient diagnosed with lung cancer, whereas African Americans only require ˜50 eligible patients for every diagnosed case of lung cancer^{9,10}. In other words, African Americans have up to double the incidence of lung cancer based on screening criteria derived from the National Lung Cancer Screening Trial composed of >90% Whites^{8,10,11}. In effect, the lower number needed to screen means that African Americans at risk for lung cancer receive a disparately lower screening benefit as compared to whites and therefore present with later stage lung cancer carrying a high degree of mortality¹². For example, the five-year survival of a Stage IV lung cancer diagnosed on symptomatic presentation is <10% as compared to >90% from a LDCT diagnosed Stage I tumor¹².

In addition to screening and survival disparities, it is increasingly evident that environmental factors, social factors, and chronic stress are associated with increased incidence of lung cancer¹³⁻¹⁵. In lung cancer, smoking is a known causative risk factor per the surgeon general's report¹⁶ however in African Americans it is known that lung cancer occurs at a higher incidence when normalized to tobacco exposure as compared to Whites^{10,11}. This finding suggests potential of ancestral, environmental, and social determinants of increasing lung cancer beyond tobacco exposure in African Americans and other minority or underserved populations. To improve health equity in lung cancer survival in African Americans and other minority and underserved populations, we hypothesized that a personalized risk assessment of cancer using combined imaging and clinical features would facilitate adoption and adherence to LDCT screening, and potentially justify broader screening eligibility criteria beyond USPSTF standards which underserve African Americans, for example.

Thus, there is a need to detect cancer earlier, where disparities are mitigated, thereby improving staging and risk. Further to this is improving these anticipated improvements in survival by enabling a clear benefit-risk window for pharmacoprevention which requires technology that improves precision to maximize the benefit to risk ratio. Therefore, the use of a multimodal AI model combines the strengths of different Artificial Intelligence (AI) techniques to achieve enhanced performance in computer vision tasks. In combination, clinical features including, for lung cancer, age, race, education, BMI, presence of COPD, personal history of cancer, family history of lung cancer, smoking status, cigarettes per day, duration of smoking, and duration of quitting or in the case of breast cancer; age, gender, family history of breast cancer, presence of genetic disposition etc. Of course, those of skill in the art will appreciate that each specific cancer may be associated with different clinical features for staging and prognosis. Together, multimodal AI and clinical features allows the clinician to better predict the risk of cancer, staging and pharmaceutical intervention of patients at risk of developing cancer and benefits of prophylactic treatments

### SUMMARY OF THE INVENTION

Disclosed is a method and process to use of artificial intelligence in methods of predicting and treating cancer with prophylactics/preventative agents. As compared to the current state of the art of clinical risk models or calculators for cancer risk and preventive measures, this invention integrates risk via artificial intelligence inference of medical imaging cancer screens. The end result is less false-positives when one is predicted to be high risk for developing cancer, thus lowering the number needed to treat for a positive outcome of cancer prevention with a prophylactic.

Therefore, disclosed herein is a method to decrease bias in identifying the risk of cancer by using a multi modal artificial intelligence model and minimizing false positives to increase benefit of administering pharmacoprevention, the method comprising: receiving, by an analysis device, one or more instances of radiographic image or images of an at risk organ for a subject; i) inputting, by the analysis device, a radiographic image or images of a patient to a classification neural network; ii) identifying by recursive feature elimination the clinically significant features and subdividing the data into patients who have clinically significant features and patients who do not; iii) inputting the subject's clinical features to the classification neural network; and iv) predicting, by the analysis device, a probability of developing cancer for the subject, wherein the classification neural network is trained using a cohort of radiographic images and/or clinical features, whereby bias is decreased. In various embodiments the cancer is lung cancer, breast cancer, colon cancer, abdominal cancer, liver cancer, kidney cancer, brain cancer, head and neck cancer, prostate cancer and pelvic cancer.

In some embodiments, the radiographic image or images comprise computed tomography images or magnetic resonance imaging. In other aspects, the neural network comprises a residual neural network, first classification network, a convolutional neural network a diffusion neural network, a multi-modal neural network, a radial basis function neural network, a recurrent neural network, a graphical neural network, a generative adversarial neural network, a vision transformer. In various embodiments, the clinical features comprise one or more of: history of cancer, genetics, plasma, serum, blood, urine or sputum concentrations of C-Reactive Protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting, mammogram results and social determinant factors. In other embodiments, predicting comprises generating a numerical risk factor between 0 and 1, where a risk factor greater than about 0.015 is predictive of developing lung cancer within 0.5-10 years.

In yet other embodiments disclosed is a method of treating a subject at enhanced risk of developing lung cancer comprising: a) determining whether the subject is at enhanced risk of developing cancer using the artificial intelligence model of claim 1; and b) administering to the subject an effective pharmacopreventive prophylactic amount of an IL-1 signaling pathway antagonist. In these embodiments, the IL-1 signaling pathway antagonist comprises: anakinra, MCC950 (CP-456773), CY-09, Oridonin, Tranilast, MNS, OLT1177 dapansutrile, Bay 11-7082, BOT-4-one, Parthenolide, and INF39, rilonacept, VX 765, ILIRAP, nadulonimab, a JAK inhibitor. In aspects the IL-1 signaling pathway antagonist comprises an antibody to human IL-1b. In some aspects the antibody comprises canakinumab. In aspects, wherein the antibody comprises a sweeping antibody. In aspects, the IL-1 signaling antagonist comprises an inhibitor to the IL-1 receptor. In aspects, the IL-1 inhibitor comprises anakinra. In aspects, the IL-1 signaling pathway antagonist comprises a JAK inhibitor. In aspects, the JAK inhibitor comprises ruxolitinib, tofacitinib, oclacitinib, baricitinib, peficitinib, upadacitinib, fedratinib, delgocitinib, filgotinib, abrocitinib, pacritinib, deucravacitinib, ritlecitinib, or momelotinib. In aspects, the cancer is lung cancer, breast cancer, colon cancer, abdominal cancer, liver cancer, kidney cancer, brain cancer, prostate cancer, head and neck cancer and pelvic cancer. In aspects, the IL-1 signaling pathway antagonist comprises an NLRP3 inflammasome inhibitor. In aspects, the NLRP3 inflammasome inhibitor comprises mcc950 (cp-456773), cy-09, oridonin, tranilast, mns, olt1177 (dapansutrile), bay 11-7082, bot-4-one, parthenolide, inf39.

However, an existing imaging model to predict lung cancer, Sybil, was unable to perform well in different groups based on race and BMI (FIG. 8-10). We constructed a multi-modal artificial intelligence model using support vector machines (SVM) and residual neural network (ResNet) predictions to improve accuracy and equivalent performance regardless of race, sex, ethnicity, or BMI. This is due to the artificial intelligence model's incorporation of these and other features to regularize the model's prediction of lung cancer, and understanding sources of bias a priori by determining significant features predictive of cancer risk and subdividing patients within different categories of these features. Further, the model can make predictions for subjects within multiple sites (NLST data and our site at our health system, University of Illinois Chicago (UIC) (see figures and tables).

In embodiments, disclosed is a method for determining the efficacy of a cancer treatment or treating agent using a multi modal artificial intelligence model, the method comprising: a) administering the treatment or treating agent to a test subject; b) receiving, by an analysis device, one or more instances of radiographic image or images of the lungs for a subject; c) inputting, to the analysis device, the radiographic image or images of the lungs to a first classification neural network; d) optionally inputting the subject's clinical features comprising history of cancer, genetics, plasma, serum, blood, urine or sputum concentrations of C-reactive protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting and social determinant factors of health; and e) assigning, by the analysis device, a numerical value indicative of the efficacy of the treatment or treating agent, wherein the first classification neural network is trained using a cohort of radiographic images of the lungs and/or clinical features. In aspects, the cancer comprises lung cancer, breast cancer, colon cancer, abdominal cancer, liver cancer, kidney cancer, brain cancer, prostate cancer, head and neck cancer and pelvic cancer, head and neck cancer.

In aspects pharmacoprevention for breast cancer comprises: a) determining whether the subject is at enhanced risk of developing breast cancer using the artificial intelligence model of claim 1; and b) administering to the subject an effective prophylactic amount of an estrogen receptor modulator or antagonist comprising tamoxifen, toremifene, raloxifene, bazedoxifene, anastrozole, letrozole, exemestane, fulvestrant, elacestrant, giredestrant, camizestrant, lasofoxifene, rintodestrant, imlunestrant, op-1250, 1sz102; and/or c) administering to the subject an effective prophylactic amount of a GLP-1 analog comprising semaglutize, tirzepatide; and/or d) administering to the subject an effective prophylactic amount of a GLP receptor agonist comprising orforglipron; wherein the progress of the cancer is monitored by the artificial intelligence model disclosed above.

In embodiments, when the cancer is prostate cancer: a) the input features comprise prostate-specific antigen test, rectal exam, urination problems or biopsy and MRI scan; and b) the clinical features comprise age, family history of prostate cancer and, obesity. In these embodiments pharmacoprevention comprises: a) determining whether the subject is at enhanced risk of developing breast cancer using the artificial intelligence model of claim 1; and b) administering to the subject an effective prophylactic amount of an radiation therapy or radiopharmaceutical therapy, hormone therapy, chemotherapy, immunotherapy, bisphosphonate therapy; wherein the progress of the cancer is monitored by the artificial intelligence model.

In embodiments disclosed is a method that reduces false positives in cancer prediction by incorporation of medical imaging into a multimodal AI model to improve precision comprising: a) incorporating a computer vision inference of medical imaging performed for cancer screening; b) incorporating into the computer vision model a multimodal AI framework a support vector machine model or decision tree model; c) determining a positive predictive value and sensitivity cutoff appropriate for clinical benefit of prophylactic intervention to adverse effects from prophylactic; d) wherein the method incorporates the computer vision inference from one medical imaging study, or a series of medical imaging studies to improve sensitivity and specificity.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1: A flowchart representing the general formula for creating a multimodal model which combines imaging and clinical features to predict an outcome. The initial data set is comprised of both imaging and clinical data which are associated with each other. First, imaging data is used to train a neural network (NN) to make predictions from imaging information only. Then, a subset of clinical features are chosen using recursive feature elimination to select for the most predictive features. The clinical features are pooled with the prediction of the imaging NN to make a final set of features. These features are then used to train a support vector machine to make a final prediction. A held-out test set is then used for evaluation of the final prediction pipeline. For a given test sample, first the imaging component is passed to the NN, and this result along with the specified clinical features are passed to the SVM to produce a final prediction.

FIG. 2. Schema of support vector machine clinical feature risk model. A) Support Vector Machine (SVM) model schema to predict lung cancer risk using NLST clinical and demographic data. B) 7-feature SVM performance assessed by ROC curves in the NLST (left) and UIC cohorts (right).

FIG. 3: Schema of identifying clinical features by optimization of support vector machine

FIG. 4 Comparison of the PLCOₘ₂₀₁₂ optimized for 6 year prediction only, versus 11 feature SVMs optimized for multiyear prediction. Top—PLCOₘ₂₀₁₂ model tested on NLST. Bottom—SVM model tested on NLST. Left panels—Precision Recall curves. Right Panels—Receiver Operating Characteristic Curves. Bottom—A table listing statistics representing the performance of the model.

FIG. 5: Seven feature SVM model tested on NLST. Left panels—Precision Recall curves. Right Panels—Receiver Operating Characteristic Curves. Bottom—A table listing statistics representing the performance of the model.

FIG. 6: Seven feature SVM model tested on UIC cohort. Left panels—Precision Recall curves. Right Panels—Receiver Operating Characteristic Curves. Bottom, A table listing statistics representing the performance of the model.

FIG. 7: Training and testing schematics and performance of the clinical and multimodal models in the NLST and UIC cohorts. A) Multimodal model (SVM+ResNet18) schema combining LDCT scans with clinical and demographic data to predict lung cancer risk. B) Multimodal model performance assessed by ROC curves in the NLST. (left) and UIC cohorts (right).

FIG. 8 Flowchart schema for the UIC cohort.

FIG. 9: Performance of the multimodal AI model on LDCTs which belong to patients in the NLST cohort. Images for which a future cancer event is unknown were removed. A) Multiple precision recall curves representing performance in correctly classifying low dose computed tomography (LDCT) images of the chest by whether they precede a lung cancer diagnosis event by a maximum of 1 to 6 years. E.g. the year 1 curve represents the model's performance when classifying an image by whether it precedes a lung cancer diagnosis event by a maximum of 365 days. B) Multiple receiver operating characteristic curves. (C) A table listing statistics representing the performance of the model. A cutoff was selected by setting the sensitivity at 0.8 to capture 8096 of lung cancer diagnosis events.

FIG. 10: Performance of the multimodal AI model on LDCTs which belong to patients in the UIC cohort. Images for which a future cancer event is unknown were removed. (A) Multiple precision recall curves representing performance in correctly classifying low dose computed tomography (LDCT) images of the chest by whether they precede a lung cancer diagnosis event by a maximum of 1 to 6 years. E.g. the year 1 curve represents the model's performance when classifying an image by whether it precedes a lung cancer diagnosis event by a maximum of 365 days. (B) Multiple receiver operating characteristic curves. (C) A table listing statistics representing the performance of the model. A cutoff was selected by setting the sensitivity at 0.8 to capture 8096 of lung cancer diagnosis events.

FIG. 11: Schematic of cloud based infrastructure for use of the invention in a healthcare setting. The patient and the provider discuss lung cancer screening and the provider places a request to the Health Care IT Network to pull clinical data and relevant imaging study (e.g. Low dose CT for lung cancer screening or other types of medical imaging study or series of studies for lung cancer or other cancers) where it is processed in a cloud infrastructure on persistent or both persistent and non-persistent memory. Alternatively, the software in the cloud infrastructure including the invention in a processing and communications device can be run locally within the Health Care IT Network or locally on the provider's computer which may be inclusive of both processing and communications functions. Data from the Health Care IT Network can be stored in the Cloud infrastructure in permanent or impermanent memory. Healthcare data could include not only the clinical data and the imaging but also corresponding radiology reports for the medical imaging component of the invention.

FIG. 12: Schematic of a device incorporating the invention. The overall device consists of a processing unit such as a Central Processing Unit with or without Graphics Processing Units or Tensor Processing Units. The device also has the ability to connect with networks using a Network Interface if the device is not being run on local hardware e.g. cloud based software. There is a user interface which may be a terminal if the device cannot be accessed on local hardware or the device itself can have input output devices and a display. The device also has storage or permanent memory which can consist of magnetic disk, optical disc, flash memory or non-volatile storage components. The Memory has the Operating System, Network Module to communicate if not running locally, and also contains the Patient Data used by the Invention including Clinical Data, Imaging Data, and optionally Laboratory Data. The Memory and Operating System also contains the Multimodal AI of the Invention and an optional Reporting module to take results and format them appropriately for Electronic Health Records or for disclosure to the patient or provider.

### DETAILED DESCRIPTION OF THE INVENTION

This invention relates to methods for improving the precision for a classification or prediction of a patient's high risk of cancer. It describes the integration of medical imaging from cancer screening tests into the current method of using clinical risk features for cancer risk assessment such as the Gail and Tyler Cuzick model for breast cancer^{17,18} and the PLCOₘ₂₀₁₂ and Bach models for lung cancer^{19,20}.

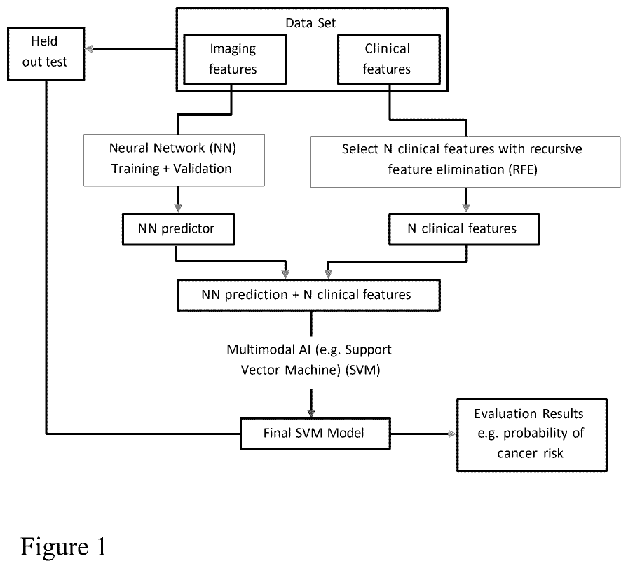

This invention relates to methods for determining a probability of developing lung cancer and to methods of treating or preventing lung cancer. This invention predicts the probability of developing lung cancer by analyzing medical images to infer lung cancer risk and integrate that risk with certain of a test subject's clinical features as defined herein. As described herein radiographic image or images of the lungs and the and/or the subject's clinical features are analyzed using a machine learning model. As is widely known, there are various types of machine learning models. For the invention described herein, it is assumed that images and clinical features are analyzed using a neural network. A deep learning network may be used as the neural network. More particularly, the neural network may be a residual neural network. Representative neural networks may include a diffusion neural network, a radial basis function neural network, a recurrent neural network, a generative adversarial neural network, transformers, vision transformers, vision language transformers, and the like, FIG. 1.

### Definitions

The following definitions are included to provide a clear and consistent understanding of the specification and claims. Unless defined otherwise, technical and scientific terms used herein have the same meaning as commonly understood by one of ordinary skill in the art to which the present disclosure belongs.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment). For example, and effective amount of buffering agent may comprise combining a biological sample and the buffering agent in a ratio of about 1:3 w/w to about 3:1 w/w, and an effective amount of non-ionic detergent may comprise a final concentration of about 0.25% to about 1% w/w, or about 0.5% w/w.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

As used herein, a device that analyzes an image using an artificial neural network is called an analysis device. The analysis device analyzes the image using a pre-trained neural network. The analysis device may derive an analysis result by inputting a medical image and/or a subject's clinical features to the artificial neural network. The analysis device may perform certain data processing or signal processing. For example, the analysis device may be implemented as a device such as a personal computer (PC), a smart device, or a server.

As used herein the term “pharmacoprevention”, also known as chemoprevention or chemoprophylaxis, is the use of medications to prevent disease or infection. It involves administering drugs to healthy individuals or those with pre-existing conditions to reduce the risk of developing a specific disease, such as cancer, or to prevent recurrence of infections. With specific respect to cancers It's often employed for individuals at high risk due to family history, specific genes, or past health issues. Pharmacoprevention can be primary (preventing cancer in a healthy person), secondary (preventing a precancerous area from becoming cancer), or tertiary (preventing recurrence after a cancer diagnosis).

As used herein Recursive Feature Elimination (RFE) refers to a feature selection technique that iteratively removes features to identify the most relevant ones to optimize a machine learning or deep learning model. It works by training a model, ranking features based on their importance, and then eliminating the least important ones. This process is repeated until the desired number of features is reached. RFE is a valuable tool for reducing model complexity, improving accuracy, and enhancing interpretability. In this invention, RFE is used to also identify a priori potential sources of bias in multimodal AI models, as clinically relevant features of race, for example, would require analysis of image based inference subdivided by race to ensure adequate representation of race associated features in training data.

Several types of imaging tests are used to detect and stage cancer, including CT scans, MRIs, X-rays, mammograms, ultrasounds, and nuclear medicine scans. CT (computed tomography) and MRI (magnetic resonance imaging) are both used to diagnose and stage cancer. CT scans are particularly good for detecting cancers in the lungs, colon, and abdominal organs like the liver and kidneys. MRI scans are often preferred for diagnosing brain tumors, prostate cancer, and certain cancers of the pelvic organs.

As used herein the term “sweeping antibody” refers to a recycling antibody that was engineered further to bind to Fc receptors and enhance the cellular uptake of antibody-antigen complex. This allows the antibody to not only bind to target antigens in plasma but also eliminate or remove them. In the context of this disclosure, the sweeping antibody comprises modifications of canakinumab to lower binding in the variable, antigen binding region in low pH environments, also known as a sweeping antibody

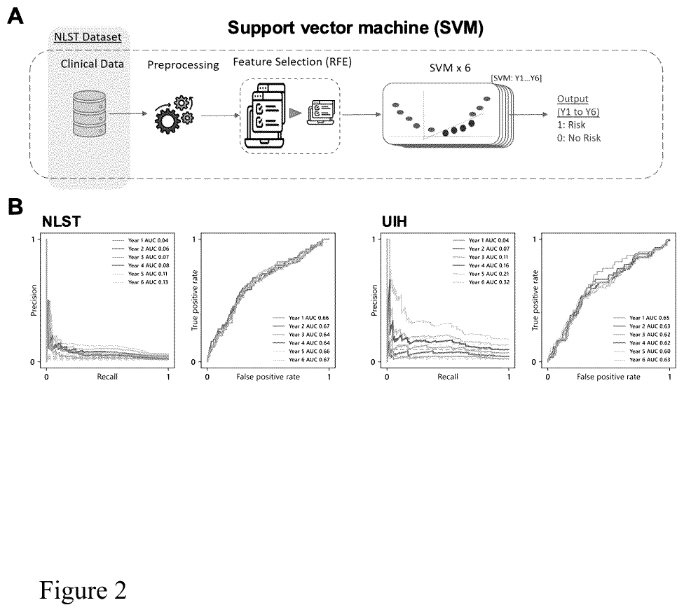

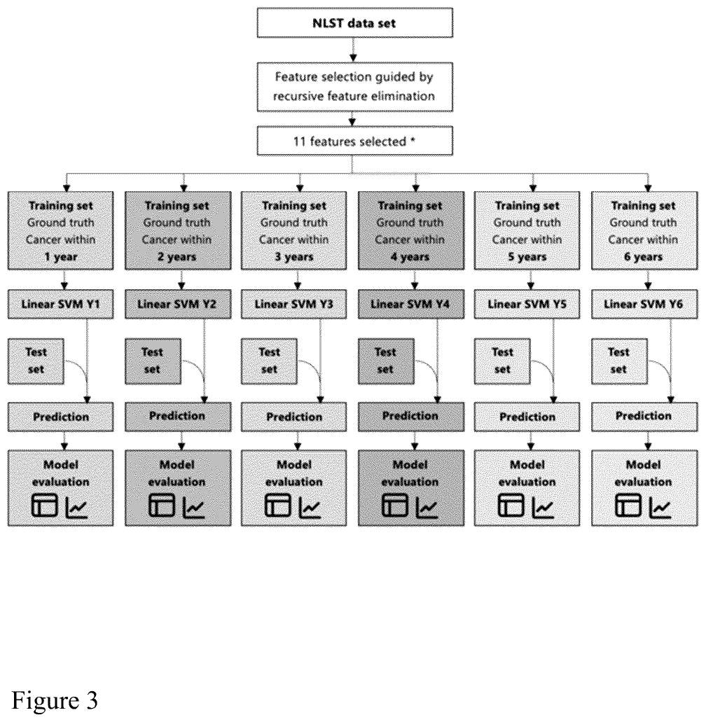

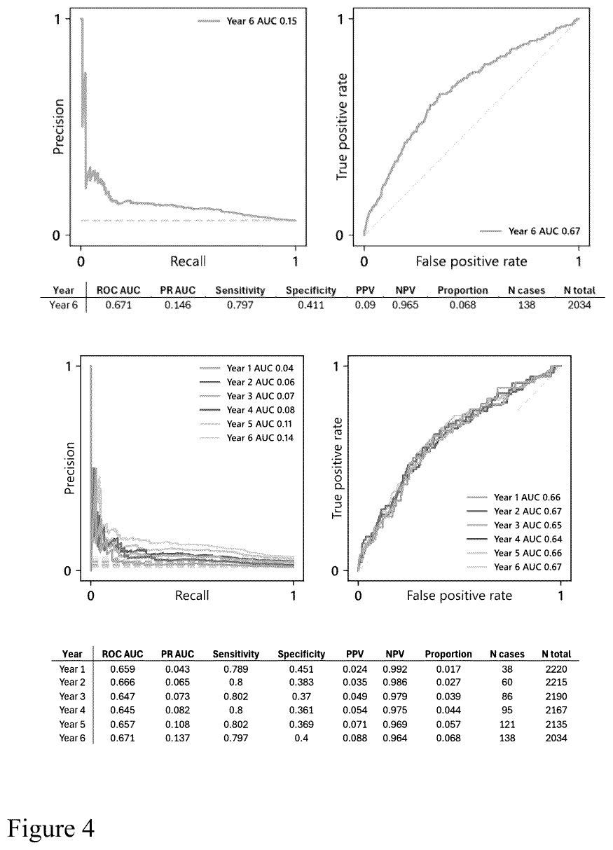

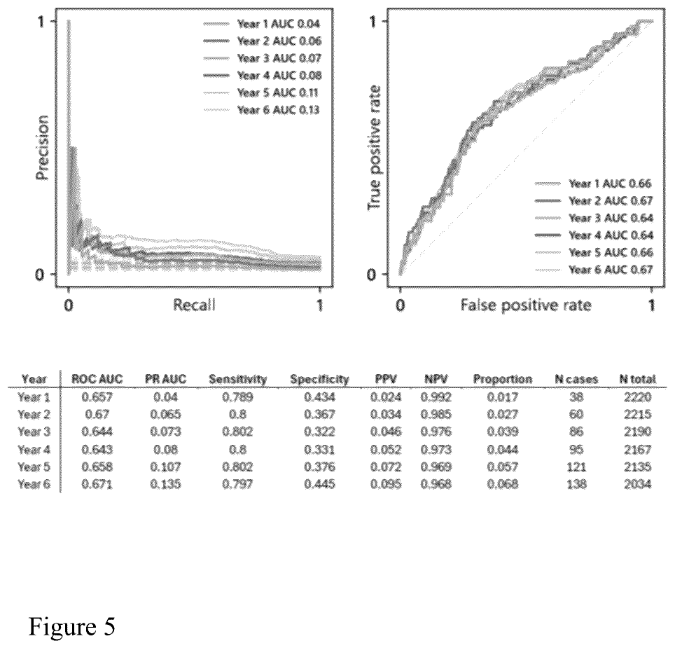

We sought to develop a multimodal AI model to combine the long-term predictive potential of clinical features as well as near-term predictive potential of radiographic features from LDCT (FIG. 1). To develop a multimodal lung cancer prediction model, we used the NLST as a training data set inclusive of CT scans and Chest X-Ray arms amounting to 48,628 total participants⁸ for the training of the support vector machine (SVM). A SVM model was chosen owing to its interpretability of features and weights and allowed for multi-year risk prognostication from baseline characteristics of participants at entry (FIG. 2). The SVM was also selected as it is effective at classifying two classes (i.e. those with no lung cancer diagnosis versus those with lung cancer diagnosis) where ground-truth was measured by the time from study entry to cancer incidence within the window of that year's specific model (e.g. SVM₁ predicts the probability of lung cancer within one year or less, SVM₂ within two years etc.). We first focused on and initiated the SVM models with 65 features available in the NLST data and utilized recursive feature elimination to optimize the model to 11 features (FIG. 3). These 11 features overlapped with many previous features identified in the PLCOₘ₂₀₁₂ 6-year risk model^{11,19} but enabled the SVM to predict multi-year risk as opposed to single 6-year risk. We then further optimized the model for practical application by eliminating features not captured in the UIC electronic health record during routine clinical care. The excluded features included former vs current smoker, whether participants have a specific family history of lung cancer, participants' education whether higher than bachelor's or not, and the maximum number of years exposed to one of the following at work: asbestos, chemical, sand blasting, coal, foundry. We found that the SVM model could be reduced to a pragmatic 7 clinical feature set while still retaining predictive performance in the NLST cohort. Performance of the 7-feature multiyear SVM model was first evaluated in a held-out test set from the NLST cohort with ROC-AUC values of 0.64-0.67 similar to the 6-year lung cancer risk when compared to the PLCOₘ₂₀₁₂ model (FIG. 4). We then applied the model to the UIC cohort and found similar performance with ROC AUC 0.60-0.65 across years and comparable Year 6 predictive performance when compared to the PLCOₘ₂₀₁₂ and 11 feature SVM (FIGS. 4 and 5).

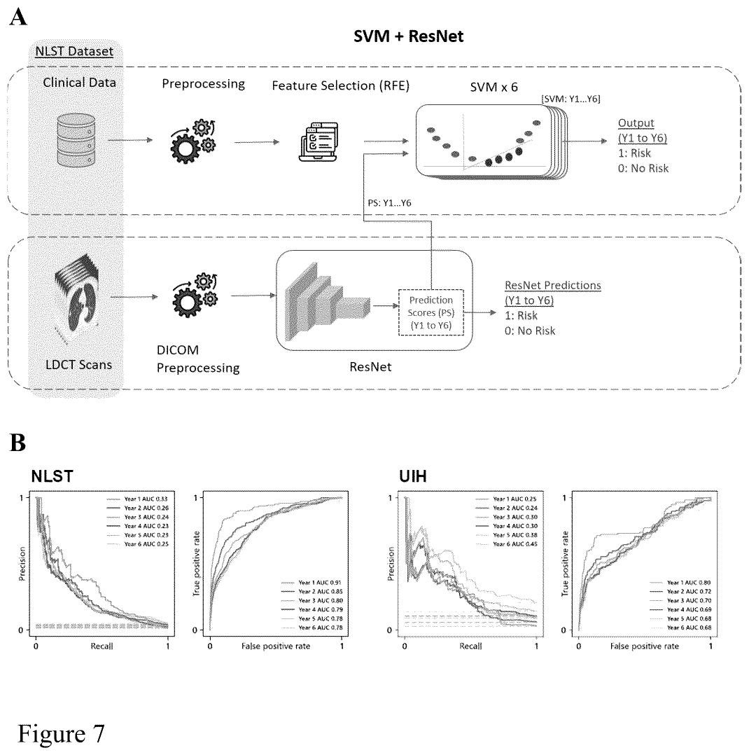

The model was trained on >40,000 participants in the National Lung Screening Trial linking clinical features at baseline with inference of lung cancer risk. Low Dose CT radiographs using a Resnet18 based classifier “Sybil 12” To evaluate the potential utility of the multimodal model, we assembled a real world cohort of >10,000 patients treated at an urban academic medical center in Chicago. This cohort, including >5,000 African American patients with history of smoking and/or lung cancer, was further split into a LDCT screening scenario and a diagnostic CT scenario as seen in Emergency Room encounters. The multimodal model demonstrated utility of identifying individual patients at imminent risk of neoplasm with high accuracy within one year and modest accuracy for 5-6 years (FIG. 7). Overall, the multimodal model demonstrates utility whereby health systems may identify individuals for enhanced follow up, lung cancer screening, and smoking cessation and adult health maintenance interventions to reduce lung cancer mortality.

However, the existing model, Sybil, was unable to perform well in different groups based on sex, race, and BMI. We constructed a multi-modal artificial intelligence model using support vector machines (SVM) and residual neural network (Resnet) predictions to improve accuracy and performance (FIG. 1). This is due to the artificial intelligence model's incorporation of these and other features to regularize the model's prediction of lung cancer. Further, the model can make predictions for subjects within multiple sites (NLST data and our site at our health system, University of Illinois Chicago (UIC) (see figures and tables).

Lastly, presented is a method of use of canakinumab and other anti IL-1 and inflammation pathway inhibitors which have been implicated in lung cancer oncogenesis^{21,22}. However, administering canakinumab (which was shown to have a hazard ratio of approximately 0.3 compared to placebo in a prospective randomized placebo controlled trial broadly to the general population or even the smoker population is impracticable as nearly one hundred asymptomatic individuals would have to be treated to prevent one lung cancer. Our artificial intelligence model is able to identify asymptomatic individuals at high risk for lung cancer and can narrow the at risk population with greater precision, or lower false positives, than conventional clinical risk models. Therefore, one would have to treat less numbers of patients with canakinumab and other anti IL-1 or other anti-inflammatory agents to prevent one lung cancer. In other words, the Artificial intelligence model holds greater yield/efficiency as compared to existing clinical models.

Importantly, the artificial intelligence algorithm takes into account clinical information about patients to refine the risk, and largely retain the preventive/prophylactic power of canakinumab and other anti IL-1 or anti-inflammatory agents while reducing the number of patients who would not benefit from this prophylaxis by incorporating imaging features which reduces false positives, or increasing precision (FIG. 7). The end result, depending on the sensitivity, or number of individuals one wishes to treat who will inevitably develop lung cancer, is that the number needed to treat is reduced from the general population level (the impracticable one) to a precision population several fold less (e.g. number needed to treat can be 5, 10, 20, 50) to prevent one lung cancer.

Magnetic Resonance Imaging of the lungs comprise Xenon-129 contrast Magnetic Resonance Image. In an embodiment, the neural network comprises a convolutional neural network. In an embodiment, the neural network comprises a residual neural network. In an embodiment, the neural network comprises a diffusion neural network. In an embodiment, the neural network comprises a radial basis function neural network. In an embodiment, the neural network comprises a recurrent neural network. In an embodiment, the neural network comprises a generative adversarial neural network. The neural network comprises a vision transformer. In an embodiment, the neural network comprises a vision language transformer. In an embodiment, the neural network comprises a graphical neural network. In an embodiment, the clinical features further comprise one or more of plasma, serum, blood, urine or sputum concentrations of C-Reactive Protein, carcinogen embryonic antigen, cell-free DNA or RNA and chemical modifications thereof, residential history, environmental exposures, and social determinant factors of health. In an embodiment, the predicting comprises generating a numerical risk factor between 0 and 1, where a risk factor greater than about 0.015 is predictive of developing lung cancer within 1-10 years.

Also approached is a method of treating a subject at enhanced risk of developing lung cancer comprising i) determining whether the subject is at enhanced risk of developing lung cancer using the artificial intelligence model of the invention; and ii) administering to the subject an effective prophylactic amount of an IL-1 signaling pathway antagonist. In an embodiment, the IL-1 signaling pathway antagonist comprises an antibody to human IL-1β. In an embodiment, the antibody comprises canakinumab. In an embodiment, the antibody comprises modifications of canakinumab to lower binding in the variable, antigen binding region in low pH environments, also known as a sweeping antibody²³. In an embodiment, the IL-1 signaling antagonize comprises an inhibitor of the IL-1 receptor and/or its subunits. In an embodiment, the IL-1 receptor antagonist comprises a polypeptide. In an embodiment, the polypeptide comprises anakinra. In an embodiment, anakinra is modified to increase IL-1 receptor by directing IL-1R to the lysosome for degradation by conjugation with CI-M6PR. Anakinra is modified to engage with a polypeptide binding moiety to engage RNF43. In an embodiment, the polypeptide binding moiety is an antibody. In an embodiment, the IL-1 signaling pathway antagonist comprises a soluble IL-1 Receptor trap to human IL-1β or IL-1α. In an embodiment, the soluble IL-1 Receptor trap comprises rilonacept.

IL-1 signaling pathway antagonist comprises an inhibitor to IL-1β or IL-1α secretion. In an embodiment, the inhibitor to IL-1b or IL-1 a secretion comprises a caspase-1 inhibitor. In an embodiment, the caspase-1 inhibitor comprises VX-765. In an embodiment, the IL-1 signaling pathway antagonist comprises an inhibitor to ILIRAP. In an embodiment, the inhibitor to IL1RAP is an antibody. In an embodiment, the antibody is nadulonimab. In an embodiment, the IL-1 signaling pathway antagonist comprises a JAK inhibitor. In an embodiment, the JAK inhibitor comprises ruxolitinib, tofacitinib, oclacitinib, baricitinib, peficitinib, upadacitinib, fedratinib, delgocitinib, filgotinib, abrocitinib, pacritinib, deucravacitinib, ritlecitinib, or momelotinib, A subject at risk of developing lung cancer identified by the AI model of the invention may be treated by a pharmacopreventive amount of an IL-1 signaling pathway antagonist. In an embodiment, the IL-1 signaling pathway antagonist comprises an NLRP3 inflammasome inhibitor. In an embodiment, the NLRP3 inflammasome inhibitor comprises mcc950 (cp-456773), cy-09, oridonin, tranilast, mns, olt1177 (dapansutrile), bay 11-7082, bot-4-one, parthenolide, inf39.

In another aspect, this invention is a method for determining the efficacy of a lung cancer treatment or treating agent using a multi modal artificial intelligence model, the method comprising: i) administering the treatment or treating agent to a test subject; ii) receiving, by an analysis device, one or more instances of radiographic image or images of the lungs for a subject; iii) inputting, by the analysis device, the radiographic image or images of the lungs to a first classification neural network; iv) optionally inputting into the analysis device the subject's clinical features comprising age, race, education, BMI, presence of COPD, personal history of cancer, family history of lung cancer, smoking status, cigarettes per day, duration of smoking, and duration of quitting first classification neural network; and v) assigning, by the analysis device, a numerical value indicative of the efficacy of the treatment or treating agent, wherein the first classification neural network is trained using a cohort of radiographic images of the lungs and/or clinical features.

Multimodal AI models combine the strengths of different Artificial Intelligence (AI) techniques to achieve enhanced performance in computer vision tasks. Specifically, “computer vision” is a field of AI that enables computers to “see” and interpret images and videos like humans do. AI computer vision involves tasks like object detection, image recognition, image segmentation, and more. For example, a multimodal model might combine a Convolutional Neural Network (CNN) for feature extraction with a Recurrent Neural Network (RNN) for processing sequential data, allowing it to understand both spatial and temporal information in videos. Vision models can provide distinct benefits including improved accuracy: by combining complementary techniques, multimodal models can often achieve higher accuracy than single-model approaches; enhanced efficiency: multimodal models can be optimized for both speed and accuracy by assigning tasks to the processing unit (CPU or GPU) best suited for them; greater robustness: multimodal models can be more resilient to variations in data and challenging conditions, as they leverage multiple perspectives and approaches; flexibility and adaptability: multimodal models can be adapted to different tasks and datasets by selecting and combining the most appropriate AI techniques (Table 1).

TABLE 1
Predictive performance metrics of the multimodal model after retraining
on 70% of the UIC cohort and testing on a 30% held out set.

| | | | | Retrained | P Boot- |
|---|---|---|---|---|---|
| | | | | multimodal | strapped |
| | | n negative | ResNet | AI | Delong's |
| | Year | n positive | ROC-AUC | ROC-AUC | Test |
| All | 1 | 1163 | 0.68 | 0.78 | 2.9 × 10⁻⁴ |
| subjects | | 34 | | | |
| | 2 | 754 | 0.66 | 0.76 | 6.7 × 10⁻³ |
| | | 49 | | | |
| | 3 | 530 | 0.66 | 0.75 | 1.1 × 10⁻² |
| | | 52 | | | |
| | 4 | 475 | 0.65 | 0.70 | 9.1 × 10⁻² |
| | | 55 | | | |
| | 5 | 341 | 0.66 | 0.73 | 2.3 × 10⁻² |
| | | 58 | | | |
| | 6 | 239 | 0.65 | 0.79 | 5.1 × 10⁻⁷ |
| | | 60 | | | |
| Black | 1 | 751 | 0.59 | 0.67 | 1.5 × 10⁻³ |
| subjects | | 28 | | | |
| | 2 | 475 | 0.58 | 0.68 | 1.4 × 10⁻² |
| | | 43 | | | |
| | 3 | 337 | 0.59 | 0.74 | 3.2 × 10⁻⁴ |
| | | 46 | | | |
| | 4 | 289 | 0.58 | 0.70 | 4.1 × 10⁻³ |
| | | 49 | | | |
| | 5 | 173 | 0.59 | 0.70 | 2.7 × 10⁻³ |
| | | 52 | | | |
| | 6 | 123 | 0.56 | 0.74 | 3.29 × 10⁻⁸ |
| | | 54 | | | |
| non-Black | 1 | 412 | 1 | 1 | NA |
| subjects | | 6 | | | |
| | 2 | 279 | 1 | 1 | NA |
| | | 6 | | | |
| | 3 | 203 | 1 | 1 | NA |
| | | 6 | | | |
| | 4 | 186 | 1 | 1 | NA |
| | | 6 | | | |
| | 5 | 148 | 1 | 1 | NA |
| | | 6 | | | |
| | 6 | 116 | 1 | 1 | NA |
| | | 6 | | | |

TABLE 2
ROC-AUC metrics of multimodal versus 7 feature
SVM in the NLST and UIC cohorts and statistical
testing (DeLong's test with bootstrapping).

| | | | Hybrid | | |
|---|---|---|---|---|---|
| | | 7 feature | computer | | |
| | | SVM | vision | | DeLong's |
| | | ROC- | ROC- | | test |
| Cohort | Year | AUG | AUC | P value | statistic |
| NLST | 1 | 0.68 | 0.91 | 0.000 | 11.524 |
| | 2 | 0.67 | 0.85 | 0.000 | 10.231 |
| | 3 | 0.64 | 0.80 | 0.000 | 9.921 |
| | 4 | 0.64 | 0.79 | 0.000 | 9.56 |
| | 5 | 0.68 | 0.78 | 0.000 | 9.848 |
| | 6 | 0.67 | 0.78 | 0.000 | 8.929 |
| UIH | 1 | 0.653 | 0.797 | 0.000 | 9.36 |
| | 2 | 0.629 | 0.724 | 0.000 | 9.013 |
| | 3 | 0.619 | 0.696 | 0.000 | 8.918 |
| | 4 | 0.617 | 0.686 | 5.651115E−15 | 8.03 |
| | 5 | 0.598 | 0.681 | 1.55431E−15 | 8.158 |
| | 6 | 0.628 | 0.684 | 3.30846E−14 | 7.395 |

The interleukin-1 (IL-1) family of cytokines comprises 11 proteins (IL-1F1 to IL-1F11) encoded by 11 distinct genes in humans and mice. IL-1-type cytokines are major mediators of innate immune reactions, and blockade of the founding members IL-1α or IL-1β by the interleukin-1 receptor antagonist (IL-1RA) has demonstrated a central role of IL-1 in a number of human autoinflammatory diseases. Moreover, levels of IL-1β are associated with tumor initiation, invasiveness, and progression in a variety of malignancies. IL-1β is abundant at tumor sites, including lung tumors; high levels of IL-1β were found in the serum and tissues of patients with lung cancer and correlated with poor prognosis.

Canakinumab (international nonproprietary name (INN) number 8836) is a member of the family of antibodies to human interleukin 1 beta (IL-1β) disclosed in WO2002016436A2 which is hereby incorporated by reference in its entirety. Canakinumab is a fully human monoclonal anti-human IL-1 p antibody of the IgGl/k isotype, being developed for the treatment of IL-Iβ driven inflammatory diseases. It is designed to bind to human IL-1β, and thereby blocking the interaction of the cytokine with its receptors. Canakinumab was found to significantly decrease the risk for and mortality associated with invasive lung cancer in the Canakinumab Anti-inflammatory Thrombosis Outcomes Study. Ridker PM, et al. Effect of interleukin-1β inhibition with canakinumab on incident lung cancer in patients with atherosclerosis: exploratory results from a randomized, double-blind, placebo-controlled trial. Lancet. 2017; 390(10105):1833-1842.

For these indications, the appropriate dosage will vary depending upon, for example, the particular IL-1 signaling pathway antagonist to be employed, the host, the mode of administration and the nature and severity of the condition being treated. However, in prophylactic use, satisfactory results are generally indicated to be obtained at dosages from about 0.05 mg to about 10 mg per kilogram body weight more usually from about 0.1 mg to about 5 mg per kilogram body weight. The frequency of dosing for prophylactic uses will normally be in the range from about once per week up to about once every 3 months, more usually in the range from about once every 2 weeks up to about once every 10 weeks, e.g. once every 4 to 8 weeks. The IL-1 signaling pathway antagonist is conveniently administered parenterally, intravenously, e.g. into the antecubital or other peripheral vein, intramuscularly, or subcutaneously. A prophylactic treatment typically comprises administering the IL-1 signaling pathway antagonist may be administered daily, weekly, once per month to once every 2 to 3 months, or less frequently.

Pharmaceutical compositions comprising the IL-1 signaling pathway antagonist may be manufactured in conventional manner. For example, a composition of the invention may be provided in lyophilized form. For immediate administration it is dissolved in a suitable aqueous carrier, for example sterile water for injection or sterile buffered physiological saline.

Canakinumab can be administered subcutaneously or intravenously. Canakinumab can be administered in a reconstituted formulation comprising canakinumab at a concentration of 50-2.00 mg/ml, 50-300 mM sucrose, 10-50 mM histidine, and 0.01-0.1% surfactant and wherein the pH of the formulation is 5.5-7.0. Canakinumab can be administered in a reconstituted formulation comprising canakinumab at a concentration of 50-200 mg/ml, 270 mM sucrose, 30 mM histidine and 0.06% polysorbate 20 or 80, wherein the pH of the formulation is 6.5. Canakinumab can also be administered in a liquid formulation comprising canakinumab at a concentration of 50-300 mg/ml, a buffer system selected from the group consisting of citrate, histidine and sodium succinate, a stabilizer selected from the group consisting of sucrose, mannitol, sorbitol, arginine hydrochloride, and a surfactant and wherein the pH of the formulation is 5.5-7.0. Canakinumab can also be administered in a liquid formulation comprising canakinumab at a concentration of 50-300 mg/ml, 50-300 mM mannitol, 10-50 mM histidine and 0.01-0.1% surfactant, and wherein the pH of the formulation is 5.5-7.0. Canakinumab can also be administered in a liquid formulation comprising canakinumab at a concentration of 50-300 mg/ml, 270 mM mannitol, 20 mM histidine and 0.04% polysorbate 20 or 80, wherein the pH of the formulation is 6.5. When administered subcutaneously, canakinumab can be administered to the patient in a liquid form contained in a prefilled syringe, autoinjector, or as a lyophilized form for reconstitution.

### EXAMPLES

The foregoing may be better understood by reference to the following examples, methods, results and discussion, which are presented solely for purposes of illustration and are not intended to limit the scope of the invention. On the contrary, one having ordinary skill in the art would readily understand that there are other embodiments, modifications, and equivalents of the present invention without departing from the spirit of the present invention and/or the scope of the appended claims.

### Methods

Research analysis was carried out under approved UIC IRB protocols 2023-1321 and 2023-1377. The NLST is a multicenter cohort and has been previously described⁸. Cook county is racially, socioeconomically, and geographically diverse and has regions with higher rates of lung cancer, predominantly in areas populated by Black patients^{7,13,14} We assembled a real-world cohort of 11,654 participants treated in urban and suburban settings in the city of Chicago as well as greater Cook County. This population based cohort, including 6,447 Black patients with history of smoking and/or lung cancer, was filtered based on available lung/chest CT studies.

### Collecting Data: NLST

Deidentified NLST data was accessed from the National Cancer Institute Cancer Data Access System approved under approved UIC IRB Protocol 2023-1321.

### Collecting Data: UIC Lung Cancer Screening

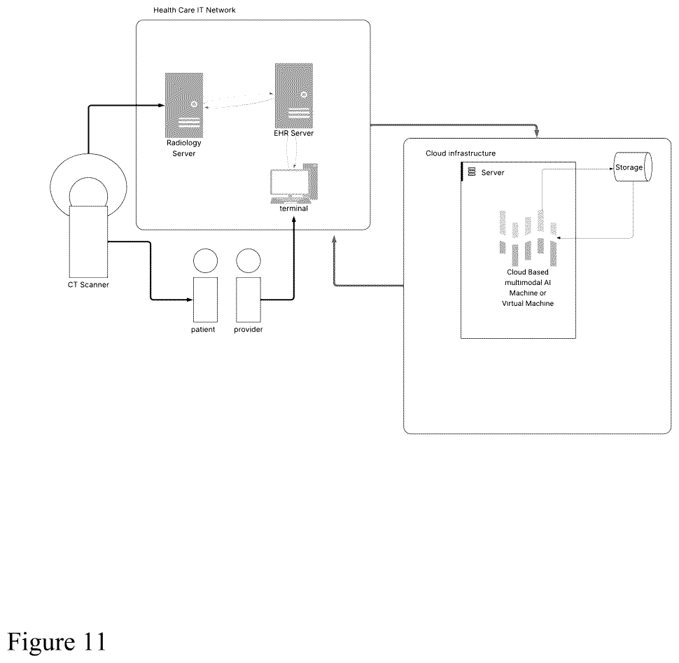

To assess the multimodal model in the UIC population, we assembled a retrospective cohort of patients with a history of smoking under approved UIC IRB protocol 2023-1377. Inclusion criteria for this cohort is listed in Table 3. Briefly, participants were placed into the cohort to mimic USPSTF lung cancer screening eligibility criteria with ages 50-80 and a history of tobacco use, and/or based on ICD codes for smoking cessation counseling and cigarette smoking, and through documented history of smoking. ICD-9 code 305.1 and ICD-10 code F17 were used to include LDCT procedures to capture participants who may have had missing data in tobacco data tables from the EHR. LDCT associated DICOMS were chosen through a filtering schema (FIG. 11). A diagnosis of lung cancer was determined based on ICD codes of neoplasm of bronchus or lung (ICD-9 162, ICD-10 C34), since 10-15 percent of patients at UIC receive stereotactic body radiation therapy without biopsy proven lung cancer. Deidentified data from positive cases were verified by A.A.S., a board-certified medical oncologist with sub specialization in thoracic oncology. Those without a diagnosis of lung cancer and engagement in the EHR during the retrospective window were assumed to have no lung cancer at the time of the preparation of this manuscript. Median duration of follow-up between LDCT scans was 431 days and the median number of LDCT scans per participant with at least one LDCT was 1 (the median is 5 if each convolution kernel and reconstruction of a given LDCT exam is counted separately).

TABLE 3
Inclusion/exclusion criteria for participants in the retrospective cohort of UIC

| Criteria | Description |
|---|---|
| Time | Jan. 1, 2015-Mar. 16, 2024 |
| Age | 50-80 as of Mar. 16, 2024 |
| Tobacco | ICD-9 305.1; ICD-10, F17; history of smoking, |
| | HCPCS: G0297, CPT4: 71271, |
| CT chest | CPT 87.4100, 87.41, 87.4102, 87.4200, 87.4101, 87.42, BP2YZZZ, |
| | BP2YYZZ, BP2Y1ZZ, BP2Y0ZZ, BP2XZZZ, BP2XYZZ, BP2X1ZZ, |
| | BP2X0ZZ, BP2WYZZ, BP2W1ZZ, BP2W0ZZ, 64490, 8E0WXBG, |
| | BW25ZZZ, BW25YZZ, BW25Y0Z, BW251ZZ, BW2510Z, BW250ZZ, |
| | BW2500Z, BW24ZZZ, BW24YZZ, BW24Y0Z, BW241ZZ, BW2410Z, |
| | BW240ZZ, BW2400Z, BR27ZZZ, BR27YZZ, BR271ZZ, BR270ZZ, |
| | B027ZZZ, B027YZZ, B027Y0Z, B0271ZZ, B02710Z, B0270ZZ, B02700Z, |
| | BB291ZZ, BB2910Z, BB290ZZ, BB2900Z, BB28ZZZ, BB28YZZ, |
| | BB28Y0Z, BB281ZZ, BB2810Z, BB280ZZ, BB2800Z, BB27ZZZ, |
| | BB27YZZ, BB27Y0Z, BB271ZZ, BB2710Z, BB270ZZ, BB2700Z, |
| | BB24ZZZ, BB24YZZ, BB24Y0Z, BB241ZZ, BB2410Z, BB240ZZ, |
| | BB2400Z, BB29ZZZ, BB29YZZ, BB29Y0Z, B320YZZ, B3201ZZ, |
| | B3200ZZ, B52SZZZ, B52SZ2Z, B52SYZZ, B52SY0Z, B52S1ZZ, |
| | B52S10Z, B52S0ZZ, B52S00Z, B52RZZZ, B52RZ2Z, B52RYZZ, |
| | B52RY0Z, B52R1ZZ, B52R10Z, B52R0ZZ, B52R00Z, B52QZZZ, |
| | B52QZ2Z, B52QYZZ, B52QY0Z, B52Q1ZZ, B52Q10Z, B52Q0ZZ, |
| | B52Q00Z, B529ZZZ, B529Z2Z, B529YZZ, B529Y0Z, B5291ZZ, |
| | B52910Z, B5290ZZ, B52900Z, B528ZZZ, B528Z2Z, B528YZZ, B528Y0Z, |
| | B5281ZZ, B52810Z, B5280ZZ, B52800Z, 78816, 78815, 78814, 72130, |
| | 72129, 72128, 71275, 71270, 71260, 71250 |
| No cancer | Prior to first imaging study, no ICD9/10 code of neoplasm 149-23/C |
| history | exception for non-melanoma skin cancers (ICD 9 173.X; ICD 10 C44.X) |

### Generating Ground Truths

Ground truths for these multiyear outputs were days from CT study to lung cancer diagnosis. The Python library “Pandas” was used in creating a data frame representing the ground truth associated with each LDCT in both the NLST and UIC data sets. The ground truth is represented by six values (1 or 0) indicating the presence or absence of a lung cancer diagnosis 1 to 6 years after a CT scan was performed. Thus, 6 sets of ground truth data were generated based on whether a. scan precedes a lung cancer diagnosis event by a maximum of [1, 2, 3, 4, 5, 6] years. The time of each CT event was compared to the time of the lung cancer diagnosis event of the associated patient. If the difference is less than or equal to a given number of years, it is labeled as a positive sample, otherwise it is labeled as a negative sample. However, if the sample has no known lung cancer diagnosis event, and the number of years exceeds the number of days to last follow-up event (or in the case of UIC data, the date 3/16/2024), then the sample is removed due to a lack of information in determining the ground truth. Over years 1-6, 1888, 3128, 3828, 4056, 4476, and 4884 studies were removed from the UIC cohort respectively (i.e. 5,980 LDCT studies initially, then 4092 with one year of follow up, etc.). The NLST data set includes the number of days to the last known cancer status of each participant, and this was used in a similar manner to remove 4, 35, 159, 280, 461, and 1082 samples (out of 11,980) from the NLST data test set for each respective year.

### Train and Test Set Selection in NLST

A training set from the NLST was created by using the same split provided by the authors of Sybil, since their model was also trained and tested on the NLST data set. Thus, when training a support vector machine using clinical and demographic features (See, FIGS. 2 and 3), the same patients used in Sybil's training set were used for the SVM, in addition to data from patients in the Chest X-ray arm of NLST. Each sample represents an individual patient. The SVM models which incorporate a ResNet (Sybil) score were also trained using the same training set, however each sample represents a CT scan associated with that patient.

### Test Set Selection in UIC

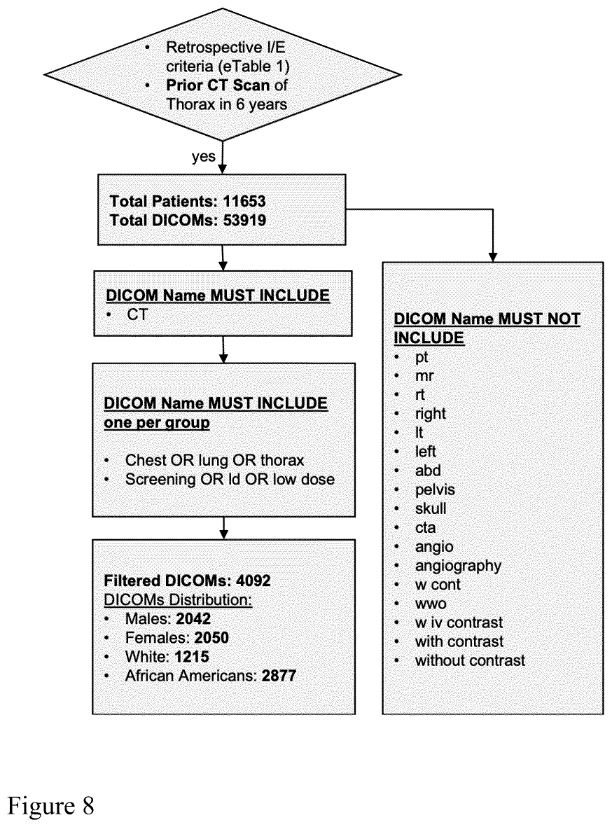

Data from the UIC cohort consisted of various Digital Imaging and Communications in Medicine (DICOM) types, a digital format for storing CT scans and associated metadata. LDCTs were selected using a schema detailed in FIG. 8.

Prediction with PLCOm2012

PLCOm2012 lung cancer risk inference was conducted on the NLST data using the PLCO adapted package (https://github.com/resplab/PLCOm2012). The PLCOm2012 model was created using a set of 11 chosen features to predict the probability of lung cancer diagnosis for a given patient within 6 years (See, FIG. 4). The 11 features are as follows: Age, Race, Education, BMI, presence of COPD, personal history of cancer, family history of lung cancer, smoking status, cigarettes per day, duration of smoking, and duration of quitting. All 11 of these values are directly represented or can be extracted from the data in the NLST. The PLCOmₘ₂₀₁₂ logistic regression model is represented in an R package (github.com/resplab/PLCOm2012). This was translated to Python code to facilitate incorporation with the code base. When using PLCOₘ₂₀₁₂ for prediction, each sample represents an individual patient. The model was used to generate predictions for probability of lung cancer diagnosis within 6 years only (it was not used to evaluate performance within 1 to 5 years) as intended by its authors, and these predictions were compared with the ground truth at 6 years, and performance was evaluated in NLST cohorts (Table 5).

TABLE 4
Benchmark metrics of PLCOₘ₂₀₁₂, SVM, Multimodal,
on NLST participants at Year 6

| Year 6 | ROC | PR | | | | | n | n |
|---|---|---|---|---|---|---|---|---|
| Model Results | AUC | AUC | Sensitivity | Specificity | PPV | NPV | positive | negative |
| PLCOₘ₂₀₁₂ Y6 | 0.671 | 0.146 | 0.797 | 0.411 | 0.09 | 0.965 | 138 | 1896 |
| 11 feat. SVM Y6 | 0.671 | 0.137 | 0.797 | 0.4 | 0.088 | 0.964 | 138 | 1896 |
| 7 feat. SVM Y6 | 0.671 | 0.135 | 0.797 | 0.445 | 0.095 | 0.968 | 138 | 1896 |
| Multimodal Y6 | 0.782 | 0.246 | 0.8 | 0.614 | 0.107 | 0.982 | 595 | 10303 |

PLCOₘ₂₀₁₂ is a logistic regression model for lung cancer risk inference and was evaluated on NLST data. The PLCOₘ₂₀₁₂ model was published in 2012, and was created using a set of 11 chosen features to predict the probability of lung cancer diagnosis for a given participant within 6 years: age, race, education, Body-Mass Index (BMI), presence of Chronic Obstructive Pulmonary Disease (COPD), personal history of cancer, family history of lung cancer, smoking status, cigarettes per day, duration of smoking, and duration of quitting. All 11 features are directly represented or can be extracted from the data in the NLST. The PLCOₘ₂₀₁₂ logistic regression model is represented in an R package created by the authors of PLCOₘ₂₀₁₂¹⁶. This was translated to Python code to facilitate incorporation with the remainder of the code base. When using PLCOₘ₂₀₁₂ for prediction, each sample represents an individual participant. The model was used to generate predictions for probability of lung cancer diagnosis within 6 years only (it was not used to evaluate performance within 1 to 5 years) as intended by its authors¹⁹.

Prediction with SVM

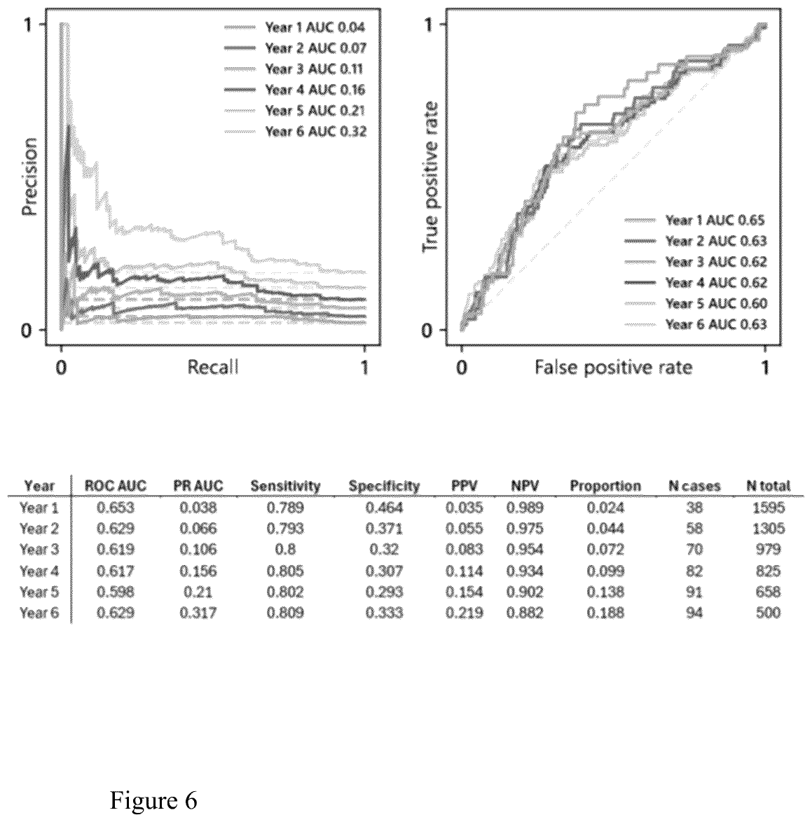

Clinical data elements from the NLST were evaluated for inclusion in a SVM using the Scikit-Learn Python library²⁴ (FIG. 3). 11 features were retained after recursive feature elimination using linear kernel. We started with a large set of initial features from those available in NLST, as well as some generated features which were calculated from existing ones, such as BMI. Recursive feature elimination showed best results when using 11 features (FIG. 2). Six unique SVMs were then trained to make predictions 1-6 years post CT scan Table 3, with each sample being an individual participant (See, FIGS. 5 and 6). Each of the 6 SVM models was then evaluated on the test sets using the corresponding ground truths. Additional SVM models were created using a truncated set of features for more practical utility using highly populated EHR fields, and to also allow for comparison between UIC and NLST cohorts where features had a high preponderance data missingness. Minimal significant differences were observed (Table 11). The training and prediction pipeline of SVM method is depicted in (FIGS. 1-3) and Features are listed in Table 5.

TABLE 5
Features and coefficients of 11 feature
SVM model trained on NLST cohort

| Feature | Coefficient |
|---|---|
| Smoking years | 0.551 |
| Age | 0.309 |
| Cigarettes per day | 0.283 |
| BMI | −0.229 |
| Divorced or widowed | 0.140 |
| Smoking status | 0.132 |
| Education: Bachelor's and above | −0.127 |
| Race: Black or African American | 0.120 |
| Family history of lung cancer | 0.102 |
| Ethnicity | −0.09 |
| Work exposure - asbestos, chemical, | 0.06 |
| sand blasting, coal mine, or foundry | |

TABLE 6
Patient characteristics of both cohorts

| Characteristic | NLST | UIC |
|---|---|---|
| Race | | |
| White | 48,549 | 3,099 |
| Black | 2,376 | 6,447 |
| Asian | 1,095 | 210 |
| American Indian or | 190 | 68 |
| Alaska Native | | |
| Native Hawaiian or | 193 | 47 |
| Other Pacific Islander | | |
| Other/No Information | 1,049 | 1,783 |
| Ethnicity | | |
| Not Hispanic | 52,118 | 9,514 |
| Hispanic | 935 | 1,170 |
| No Information | 399 | 970 |
| Lung cancer incidence | 3.2% | 8.1% |

### ResNet Prediction

DICOM data files were evaluated by a ResNet-18 CNN model, trained on the ImageNet database with parameters locked and further trained upon the NLST cohort as previously described²⁵. The ResNet model, Sybil, was run with default parameters on the same subset of the NLST cohort comprised of 11,980 LDCT studies from 2,328 participants. The resulting output from each LDCT image was a set of 6 values indicating the probability that lung cancer was diagnosed within 1 to 6 years from the date of the CT study. In the UIC cohort, the ResNet model was used on 4,092 LDCT studies from 1,104 participants. Each prediction score was then compared with the ground truth values to determine the model's performance, broken down into 6 performance evaluations by years 1 to 6.

### SVM+ResNet Model Prediction

For the multimodal AI model, SVM learning was used to train six unique models for years 1-6 post imaging including the corresponding ResNet inferred risk for that given year (FIG. 7). Each ResNet+SVM model incorporates the ResNet risk inference of the current year and that the training and testing samples are individual DICOMs associated with the clinical features of each subject ID at baseline study entry. The training and test sets used the same allocation of NLST participants used to train the previous ResNet model, and a 70:30 split of participants for the UIC training cohort. To ensure equal comparison between the various models, training and test sets had identical allocation of participants and corresponding DICOM files Table 1 and 7 resnet performance.

TABLE 7
ResNet performance comparison across races in the NLST and UIC cohorts.

| | | | | | | | | | | | | | P value |
|---|---|---|---|---|---|---|---|---|---|---|---|---|---|
| ROC-AUC | | | | | | | P value ROC-AUC (Hanley-McNeil) | | | | | | PR-AUC |
| PR-AUC | | | | | | | | NLST | NLST | NLST | NLST | UIH | (bootstrap) |
| n negative | | | | | | | | White | Black | White | White | White | NLST |
| n positive | | | | | | | | vs | vs | vs | vs | vs | White vs |
| | | | NLST | NLST | UIH | UIH | NLST | UIH | UIH | UIH | NLST | UIH | NLST |
| Year | NLST | UIH | White | Black | White | Black | vs UIH | White | Black | Black | Black | Black | Black |
| Y1 | 0.919 | 0.796 | 0.918 | 0.991 | 0.97 | 0.737 | 2.8 × | 6.1 × | 5.3 × | 1.3 × | 2.9 × | 7.4 × | 6.0 × |
| | 0.333 | 0.268 | 0.342 | 0.125 | 0.465 | 0.224 | 10 [symbol] | 10 [symbol] | 10 [symbol] | 10 [symbol] | 10 [symbol] | 10 [symbol] | 10 [symbol] |
| | 11832 | 3973 | 11234 | 290 | 932 | 2786 | | | | | | | |
| | 144 | 119 | 139 | 1 | 28 | 91 | | | | | | | |
| Y2 | 0.856 | 0.731 | 0.853 | 0.93 | 0.846 | 0.657 | 5.0 × | 8.7 × | 8.8 × | 1.5 × | 3.9 × | 4.6 × | 3.8 × |
| | 0.261 | 0.269 | 0.259 | 0.249 | 0.478 | 0.220 | 10 [symbol] | 10 [symbol] | 10 [symbol] | 10 [symbol] | 10 [symbol] | 10 [symbol] | 10 [symbol] |
| | 11702 | 2686 | 11111 | 284 | 544 | 1881 | | | | | | | |
| | 243 | 166 | 234 | 4 | 62 | 124 | | | | | | | |
| Y3 | 0.804 | 0.713 | 0.803 | 0.851 | 0.789 | 0.677 | 4.0 × | 7.1 × | 8.9 × | 2.9 × | 6.3 × | 1.1 × | 1 × |
| | 0.239 | 0.329 | 0.243 | 0.189 | 0.436 | 0.306 | 10 [symbol] | 10 [symbol] | 10 [symbol] | 10 [symbol] | 10 [symbol] | 10 [symbol] | 10 [symbol] |
| | 11479 | 1954 | 10907 | 272 | 470 | 1370 | | | | | | | |
| | 342 | 198 | 327 | 6 | 62 | 136 | | | | | | | |
| Y4 | 0.775 | 0.701 | 0.774 | 0.719 | 0.791 | 0.657 | 3.1 × | 6.9 × | 5.6 × | 6.7 × | 6.0 × | 2.3 × | 7 × |
| | 0.224 | 0.340 | 0.225 | 0.156 | 0.459 | 0.310 | 10 [symbol] | 10 [symbol] | 10 [symbol] | 10 [symbol] | 10 [symbol] | 10 [symbol] | 10 [symbol] |
| | 11255 | 1720 | 10707 | 261 | 426 | 1187 | | | | | | | |
| | 444 | 204 | 424 | 8 | 58 | 142 | | | | | | | |
| Y5 | 0.762 | 0.707 | 0.75 | 0.716 | 0.806 | 0.657 | 1.1 × | 2.0 × | 5.8 × | 3.2 × | 6.7 × | 4.0 × | 2 × |
| | 0.224 | 0.417 | 0.222 | 0.19 | 0.550 | 0.387 | 10 [symbol] | 10 [symbol] | 10 [symbol] | 10 [symbol] | 10 [symbol] | 10 [symbol] | 10 [symbol] |
| | 10972 | 1287 | 10439 | 258 | 327 | 884 | | | | | | | |
| | 547 | 217 | 522 | 8 | 68 | 149 | | | | | | | |
| Y6 | 0.752 | 0.695 | 0.752 | 0.7 | 0.782 | 0.653 | 1.7 × | 4.1 × | 6.9 × | 5.0 × | 5.7 × | 2.8 × | 0.00 |
| | 0.233 | 0.466 | 0.232 | 0.189 | 0.596 | 0.445 | 10 [symbol] | 10 [symbol] | 10 [symbol] | 10 [symbol] | 10 [symbol] | 10 [symbol] | |
| | 10303 | 886 | 9805 | 233 | 229 | 567 | | | | | | | |
| | 595 | 230 | 567 | 8 | 70 | 160 | | | | | | | |

[symbol]

indicates data missing or illegible when filed

Prediction with Sybil

DICOM data files were evaluated by a Resnet 18 CNN model, trained on the Imagenet database with parameters locked and further trained upon the NLST cohort as previously described¹². The Resnet18 model, Sybil, was run with default parameters on >10,000 individuals across the NLST training and test sets with multi-year outputs for followup. Every LDCT found in then NLST data set was evaluated using Sybil. DICOMs containing scout images were removed. DICOMS which caused errors when evaluated using Sybil were also removed, for example, DICOMs with a layer thickness above the maximum 5 mm limit. Thus, using Sybil each IC OM was associated with 6 prediction values. The first value represents the probability that the patient associated with this DICOM will have a lung cancer diagnosis event within 1 year, the second value represents the aforementioned, but within 2 years, and so forth, up to 6 years. Each prediction score is then compared with the ground truth values to determine the model's performance, broken down into 6 performance evaluations by years 1 to 6 (FIG. 8)(Tables 9 and 10.

TABLE 8
Res-Net performance metrics among individuals with varying BMI in the NLST cohort.

| | ROC AUC | | PR AUC | | Sensitivity | | Specificity | | PPV | |
|---|---|---|---|---|---|---|---|---|---|---|
| | BMI < | BMI ≥ | BMI < | BMI ≥ | BMI < | BMI ≥ | BMI < | BMI ≥ | BMI < | BMI ≥ |
| Year | 30 | 30 | 30 | 30 | 30 | 30 | 30 | 30 | 30 | 30 |
| 1 | 0.94 | 0.88 | 0.37 | 0.23 | 0.78 | 0.79 | 0.92 | 0.83 | 0.11 | 0.05 |
| 2 | 0.87 | 0.82 | 0.29 | 0.18 | 0.80 | 0.81 | 0.80 | 0.63 | 0.08 | 0.04 |
| 3 | 0.82 | 0.76 | 0.26 | 0.17 | 0.82 | 0.80 | 0.63 | 0.56 | 0.06 | 0.05 |
| 4 | 0.78 | 0.77 | 0.23 | 0.19 | 0.78 | 0.81 | 0.59 | 0.56 | 0.07 | 0.06 |
| 5 | 0.76 | 0.78 | 0.23 | 0.22 | 0.80 | 0.82 | 0.53 | 0.56 | 0.09 | 0.07 |
| 6 | 0.74 | 0.79 | 0.23 | 0.25 | 0.82 | 0.77 | 0.44 | 0.64 | 0.09 | 0.09 |

| | NPV | | n positive | | n negative | |
|---|---|---|---|---|---|---|
| | BMI < | BMI ≥ | BMI < | BMI ≥ | BMI < | BMI ≥ |
| Year | 30 | 30 | 30 | 30 | 30 | 30 |
| 1 | 0.99 | 0.99 | 102 | 42 | 8295 | 3537 |
| 2 | 0.99 | 0.99 | 176 | 67 | 8202 | 3500 |
| 3 | 0.99 | 0.99 | 243 | 99 | 8033 | 3446 |
| 4 | 0.99 | 0.99 | 326 | 118 | 7842 | 3414 |
| 5 | 0.98 | 0.99 | 415 | 132 | 7654 | 3318 |
| 6 | 0.98 | 0.98 | 455 | 140 | 7200 | 3103 |

TABLE 9
ResNet performance metrics among individuals with varying BMI in the UIC cohort

| | ROC AUC | | PR AUC | | Sensitivity | | Specificity | | PPV | |
|---|---|---|---|---|---|---|---|---|---|---|
| | BMI < | BMI ≥ | BMI < | BMI ≥ | BMI < | BMI ≥ | BMI < | BMI ≥ | BMI < | BMI ≥ |
| Year | 30 | 30 | 30 | 30 | 30 | 30 | 30 | 30 | 30 | 30 |
| 1 | 0.90 | 0.67 | 0.31 | 0.22 | 0.77 | 0.78 | 0.89 | 0.26 | 0.16 | 0.03 |
| 2 | 0.83 | 0.59 | 0.30 | 0.23 | 0.85 | 0.76 | 0.61 | 0.12 | 0.12 | 0.05 |
| 3 | 0.81 | 0.57 | 0.38 | 0.30 | 0.81 | 0.77 | 0.62 | 0.16 | 0.20 | 0.07 |
| 4 | 0.79 | 0.56 | 0.39 | 0.31 | 0.79 | 0.80 | 0.61 | 0.12 | 0.21 | 0.09 |
| 5 | 0.81 | 0.56 | 0.52 | 0.33 | 0.80 | 0.81 | 0.64 | 0.13 | 0.30 | 0.12 |
| 6 | 0.78 | 0.56 | 0.56 | 0.37 | 0.81 | 0.81 | 0.59 | 0.15 | 0.38 | 0.17 |

| | NPV | | n positive | | n negative | |
|---|---|---|---|---|---|---|
| | BMI < | BMI ≥ | BMI < | BMI ≥ | BMI < | BMI ≥ |
| Year | 30 | 30 | 30 | 30 | 30 | 30 |
| 1 | 0.99 | 0.97 | 65 | 54 | 2261 | 1712 |
| 2 | 0.99 | 0.89 | 94 | 72 | 1492 | 1194 |
| 3 | 0.97 | 0.89 | 120 | 78 | 1046 | 908 |
| 4 | 0.96 | 0.86 | 123 | 81 | 931 | 789 |
| 5 | 0.94 | 0.83 | 132 | 85 | 685 | 602 |
| 6 | 0.91 | 0.79 | 145 | 85 | 465 | 401 |

Prediction with SVM, Incorporating Sybil Score as a Feature

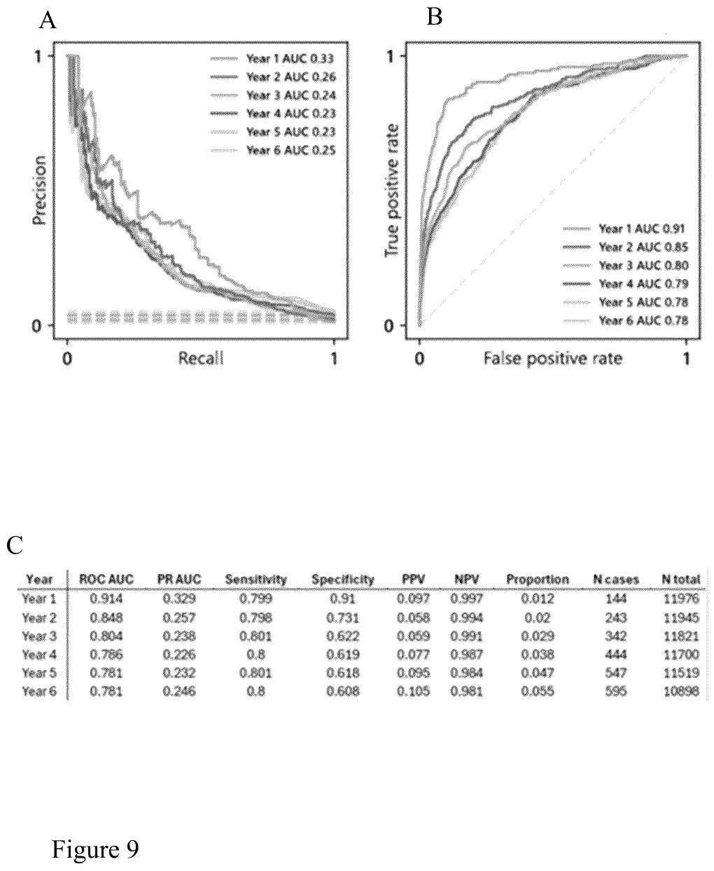

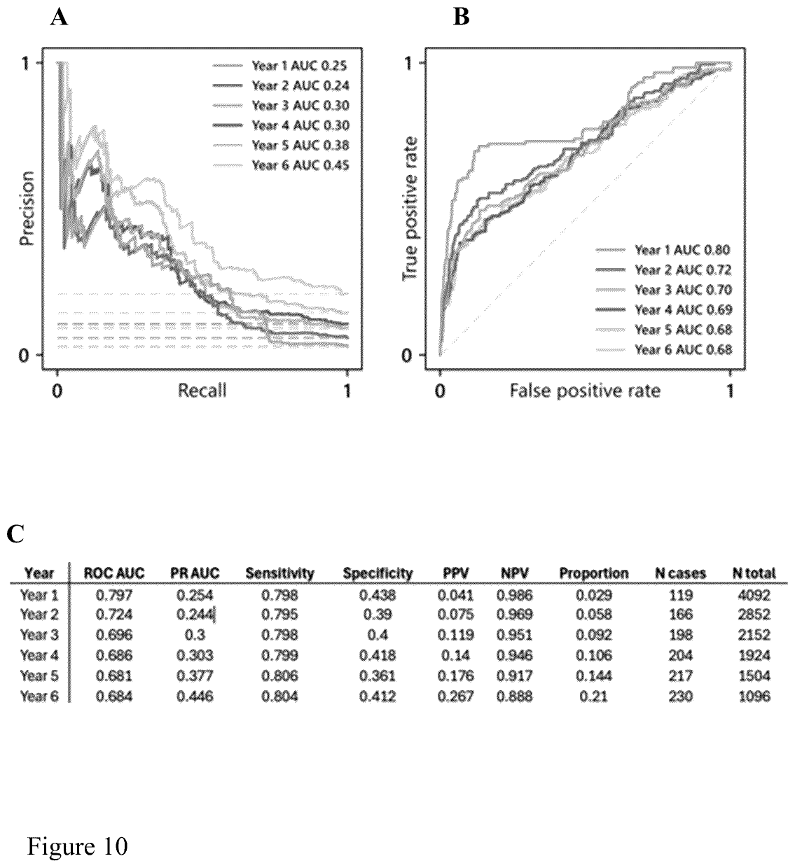

For the multimodal model, Support Vector Machine learning was used to train six unique models for years 1-6 post imaging including the corresponding CT inferred risk for that given year. Creating the Sybil+SVI models involves the same process as the initial SVI, with two main differences. Each Sybil+SVM model incorporates an additional feature: the Sybil score of the corresponding year. The other main difference is that the training and testing samples are individual DICOMs, where the clinical and demographic features are extracted from the associated patient ID, rather than each sample being a patient. The models were thus trained using the same patient ID set used for training Sybil, and the test set was also the same as the one used for Sybil. (See, FIGS. 9 and 10).

### Evaluating Model Performance

Model performance was evaluated on completely held out test sets within the NLST cohort as well as the Real World cohort. When models are tested on a given test set, both a precision-recall (PR) curve and a receiver operating characteristic (ROC) curve are generated using Scikit-Learn. Then, a prediction cutoff value is selected such that the sensitivity is as close to 0.8 as possible to capture 80% of lung cancer diagnosis events. Then, the rest of the statistics (specificity, PPV, and NPV) are calculated based on this cutoff value. In addition, model performance can be enhanced by including multiple interval scans, that is multiple monthly or yearly cancer scans will be included in the ID set for each patient.

### Comparison of Models

To determine whether there was a significant difference in the ROC curve of a given model on a similar population, DeLong's test²⁶ with a modified bootstrapping approach was utilized²⁷. For precision recall curve analysis on NLST White versus Black participants, 1000 times bootstrap resampling was performed. Unpaired analysis on ROC curves was performed as previously described by Hanley and McNeil²⁸

TABLE 10
Bootstrap resampling based DeLong's Statistics
of 7 feature SVM vs 11 feature SVM

| | | DeLong's test |
|---|---|---|
| Year | P-Value | statistic |
| Y 1 | 0.944 | −0.07 |
| Y 2 | 0.81 | 0.24 |
| Y 3 | 0.05 | 1.956 |
| Y 4 | 0.004 | 2.891 |
| Y 5 | 0.086 | 1.718 |
| Y 6 | 0.648 | −0.456 |

### Results

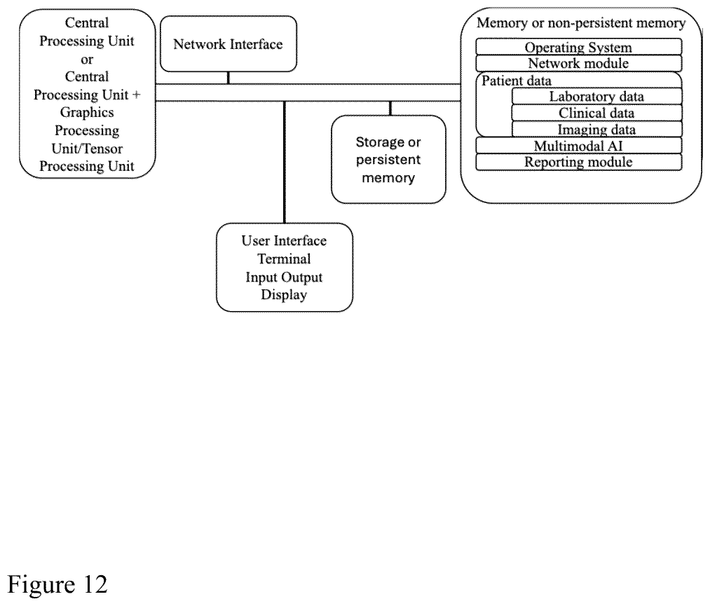

NL ST and UIC cohorts evaluated in this study and racial and ethnic breakdown initially comprised of 53,452 patients in NLST and 11,654 patients in UIC (Figures). Racial composition and ethnicity are detailed in FIG. 12. To develop a multimodal lung risk model, we used the NLST as a training data set inclusive of CT and Chest X Ray arms amounting to 48,628 patients. Support Vector Machine (SVM) was developed for multi-year risk prognostication from baseline characteristics of participants at entry (Figures) where ground-truth was measured by days from study entry to cancer incidence within the window of that year's specific model (e.g. SVM₁ predicts the probability of lung cancer within one year or less SVM₂ within two years etc.). Using recursive feature elimination with an initial set of 65 features, the final SVM was chosen to have 11 features, with feature weights listed in Table 5 validating previously identified features in the PLCOₘ₂₀₁₂ 6-year risk model developed from the PLCO cohort¹⁵. Performance of the multiyear SVM model was evaluated in a held-out test set with ROC-AUC values of 0.64-0.67. Performance was not significantly different for 6-year lung cancer risk when compared to the PLCOₘ₂₀₁₂ model (FIG. 4).

Using this approach, we then developed a multimodal risk model by incorporating a modification of the Resnet18 convolutional neural network image classifier with LDCT images from NLST, Sybil, which infers lung cancer risk probabilities at multiple years of follow up. The multimodal risk model was trained by clinical features identified by the prior SVM but with inclusion of the inference of multiyear CT image risk inference. The multimodal model was trained on 65,161 DICOMs in NLST and demonstrated substantially improved accuracy with (ROC-AUC values Y1-Y6 all significantly improved (FIG. 7, p-values <10-3, values listed in Table 2). When compared to imaging alone, the multimodal model also improved ROC-AUC, PR-AUC, and positive predictive and negative predictive values at a benchmarked sensitivity of 80% more modestly but significantly at long-term lung cancer risk at years 5 and 6 (corresponding to a PLCOₘ₂₀₁₂ 6 year risk of 15%¹6.

To assess the feasibility and utility of this multimodal model in the UI Health patient population, we assembled a retrospective Real World Cohort of patients with history of smoking under an IRB approved protocol (Table 5). Inclusion criteria for this cohort is listed by LDCT screening patients were identified by CPT code and on the basis of ICD10 codes for smoking cessation counseling, cigarette smoking, and through documented history of smoking. In addition to LDCT patients, we also expanded the Real World cohort to include patients with smoking histories and CT imaging. Diagnoses of lung cancer were made on the basis of ICD10 codes of neoplasm of bronchus or lung (ICD10 34). Those without diagnoses of lung cancer were assumed to have no lung cancer at the time of the preparation of this manuscript. The cohort overall is presented in Table 6 amounting, with 55% African American. Median duration of follow-up between LDCT scan was 1-8 years and the median number of LDCT scans per patient was 1-3, similar to what has been reported nationally.

The limitations of the Real World cohort reflected the practice of our institution including limited documentation of clinical features captured in the NLST. For example, education status, work exposure, and detailed tobacco history was limited. Patient clinical data elements that were largely representative of the Real World cohort were age, race, marital status, and tobacco history and are listed in (See, Tables 5-7). To build a multimodal model with pragmatic clinical features, we trained an SVM on the NLST cohort and tested on the UIC cohort with values ranging in 0.6-0.7 (FIGS. 5 and 6). Having confirmed modest predictive ability, we then incorporated imaging risk inference from the NLST data and tested the NLST and UIC cohorts (FIGS. 7, 9 and 10). In the UIC cohort, we confirmed predictive accuracy with R(O)C-AUC values which ranged from 0.85-0.68 for lung cancer risk Years 1-6, respectively. In contrast, PR-AUC values ranged from 0.41-0.57 progressively through Years 1-6, owing to the increased proportion of patients diagnosed with lung cancer during later years (Table 2, FIG. 10). Because the clinical feature model did not demonstrate a significant difference between NLST and UIC cohorts (FIG. 2), we evaluated the performance of the ResNet component and found bias by race and BMI (Tables 7-9). When examining performance by race, White had performance approximately ranging from >0.9 to 0.79 whereas in African Americans it ranged from approximately 0.8 to 0.7 for Years 1-6, respectively. Similar decreases in White versus African Americans were seen in PR-AUC (Table 7). Retraining the multimodal AI model by incorporating more patients from the UIC cohort, which had a higher proportion of these patients improved performance as compared to the original ResNet model (Table 1).

We also evaluated the number needed to treat with canakinumab which was published having a hazard ratio of 0.3 for the high dose cohort (300 mcg every 3 months) versus placebo 1. At various levels of sensitivity of the various risk models, we predicted the number needed to treat with a pharmacoprevention strategy to prevent one lung cancer using the formula (1/(PPV*HR_{canakinumab})) or (1/(PPV*0.3)). We found that at modest sensitivities of 50%, the number needed to treat for canakinumab ranged approximately between 5 individuals to 10 individuals across the various models, potentially paving the way to prevent lung cancers in an asymptomatic population without a need for a diagnostic classified lung pathology such as a lung mass, lung nodule, etc.

### DISCUSSION

Cancer disparities are well documented in populations, both by minority and sex, with respect to survival, outcomes, and screening. LDCT screening has been demonstrated to reduce mortality as early detection of lung cancer results in treatment of earlier stage tumors with increased survival. However, the United States Preventive Services Task Force guidelines, largely based on the NLST study population of nearly 95% Whites (Table 6) has been shown to promote disparities in lung cancer screening, especially in African Americans where the screening eligible population carries higher lung cancer risk as compared to Whites. Alternative strategies have been proposed including utilizing the PILCOₘ₂₀₁₂ criteria^{11,19} or alternative methods which demonstrate increased sensitivity as compared to USPSTF criteria, but only at long-term risk levels of 1-1.5% or greater of developing lung cancer and with no incorporation of risk adjustment on the basis of low dose CT screening findings, and limited PPV and NPV values at sensitivities >80%. Here, we evaluate a multimodal AI model capable of providing multi year risk prognosis that incorporates a new framework of both clinical elements and imaging based risk inference with validation across the NLST trial cohort and a real world cohort of diverse patients. At similar sensitivity threshold of 80% where the PILCOₘ₂₀₁₂ 6 year lung cancer risk is >1%¹⁶ the multimodal model Year 6 risk inference significantly had greater positive predictive and negative predictive values (FIGS. 7, 9 and 10) when PLCOₘ₂₀₁₂ was run on the same test set of NLST participants. Overall, the performance of the multimodal model within the NLST cohort of patients showed high accuracy and positive predictive value than clinical features alone, though a better evaluation of the multimodal model could be carried out on more inclusive cohorts such as the International Lung Screening Trial¹⁵.

When the multimodal model was evaluated in the real world cohort, we first had to adapt to the real world availability of demographic information in our institution's EJR. Despite adopting a pragmatic approach of a feature set limited to marital status, age, gender, race, ethnicity, BMI and tobacco usage, we observed consistent performance in lung cancer prediction in LDCT screening patients with minimal significant differences (FIG. 6, Table 5).

When further broken down by race and gender, we noted the multimodal model demonstrated higher performance among Whites as compared to African Americans, reflecting potential training bias in Sybil, which was trained on scans from >90% of whites. Bias in AI models is an increasing concern, particularly among institutions serving diverse patient populations and may further exacerbate health disparities^{29,32}. However many initiatives are aiming to reduce bias in AI development including Project Implicit, AIM-AHEAD³³ and specifically in lung cancer screening, CANDID-4AI³⁴. Given the existing disparities in lung cancer including the higher risk of lung cancer observed among African Americans who smoke, implementation of AI models will require representative data in training sets and inclusion of African Americans and other minorities in validation cohorts and studies.

The multimodal model also holds potential for guiding health systems and self-insured employers for implementation of LDCT screening to capture more patients at increased risk of lung cancer than the current USPSTF recommendations and CMS guidance (i.e. age >50 years and >20 pack year smoking history). One could envision patients smoking histories could have lung cancer risk inference from a single LDCT study and recommendations could then be made for formal LDCT screening and interventions such as smoking cessation. Health systems could determine risk levels suitable for specific recommendations based on positive predictive values (e.g. 1 year risk levels where PPV <0.2, recommend L)CT screening; 1 year risk levels where PPV >0.2, recommend LDCT screening and intensive tobacco cessation program). In addition to increasing enrollment in screening and prevention, the multimodal model could also increase adherence to annual follow up for LDCT, where it has been reported to be approximately 50% in the United States^{35,36}. In this scenario, patients could be informed of their specific higher than average risk of developing lung cancer which may serve to motivate them to adhere to LDCT follow up. In addition, Health Systems including safety net hospitals may flag these patients for outreach and reminders to utilize screening and smoking cessation services.

Prior methods for cancer staging and pharmacoprevention has several limitations, chiefly the limited diversity of the training set. Disclosed is a more robust and inclusive artificial intelligence models using one or more CT studies across CNN, ResNet, Diffusion, Transformers, Generative Adversarial Network models on racially diverse training data including deep clinical parameters of the 11 features and potentially more including social determinants of health and environmental features. The multimodal model also utilizes a convolutional neural network architecture trained on the NLST cohort which has implicit bias in the representation of non-minorities. Further efforts with a more inclusive training set will mitigate bias and is expected to improve generalizability and performance across health systems. Finally, the CNN architecture and risk inference has limited interpretability aside from attention mapping. A more interpretable imaging based from Vision Transformers will assist healthcare practitioners including radiologists to understand what features to evaluate on follow up studies, or to potentially implementing a dedicated diagnostic quality Chest CT within a narrower field to balance between full dose radiation exposure and LDCT on follow up.

Finally, owing to demonstrated pharmacoprevention of lung cancer with canakinumab in a broad unselected population, we describe how our artificial intelligence model and other alternative embodiments of the model would pave the way for Jung cancer pharmacoprevention. The model would classify individuals at high risk based on a selected threshold of lung cancer risk. Here, the model's sensitivity of 80% corresponded to lung cancer risk >1% across >5 years of follow up. The models have a general positive predictive value of 0.1 or a true positive rate of 10% or classifies 10 people at high risk of lung cancer with one person developing lung cancer. If one further selects a lower sensitivity threshold to 50%, one classifies approximately five individuals as high risk of lung cancer and is able to accurately detect one individual at risk of lung cancer (positive predictive value approximately 0.5, or 50% risk of lung cancer). This means if one were to treat these ten individuals at this threshold with canakinumab or another anti IL1 or anti inflammatory, one would successfully prevent at least two lung cancers since canakinumab reduces lung cancer incidence by >60% in the previous placebo controlled trial. One could envision the multimodal AI model being used as a companion diagnostic for approved medications or interventions or as a tool to select patients for clinical trial or for randomization in a clinical trial. Examples of model tuning for sensitivity and PPV to guide number needed to treat (NNT) for a clinical trial or to guide a clinician in prescribing a prophylactic, such as canakinumab, is provided in Tables 11-13 for various AI models described based on prediction years. Importantly, incorporating A image risk inference reduces false positives, improves positive predictive value and corresponding number needed to treat as compared to historical clinical only models such as the PLCOₘ₂₀₁₂ (see PR-AUC in FIG. 4, where lowering sensitivity does not have an increase in precision, or PPV as compared to FIG. 9 in NLST patients) or USPSTF criteria.

TABLE 11
Comparison between sensitivity (Sens) and positive predictive value (PPV) of the Sybil standalone model on the NLST
cohort. NNT = Number Needed to Treat to prevent one lung cancer based on number of patients based on positive
predictive value treated with an anti IL1 signaling agent with a hazard ratio similar to canakinumab hazard ratio of 0.3.

| Year 1 | | | Year 2 | | | Year 3 | | | Year 4 | | | Year 5 | | | Year 6 | | |
|---|---|---|---|---|---|---|---|---|---|---|---|---|---|---|---|---|---|
| Sens | PPV | NNT | Sens | PPV | NNT | Sens | PPV | NNT | Sens | PPV | NNT | Sens | PPV | NNT | Sens | PPV | NNT |
| 0.097 | 0.824 | 3.64 | 0.099 | 0.667 | 4.50 | 0.093 | 0.674 | 4.45 | 0.099 | 0.55 | 5.45 | 0.095 | 0.486 | 6.17 | 0.113 | 0.479 | 6.26 |
| 0.194 | 0.609 | 4.93 | 0.193 | 0.435 | 6.90 | 0.199 | 0.415 | 7.23 | 0.2 | 0.344 | 8.72 | 0.199 | 0.333 | 9.01 | 0.192 | 0.361 | 8.31 |
| 0.319 | 0.407 | 7.37 | 0.3 | 0.356 | 8.43 | 0.336 | 0.262 | 11.45 | 0.293 | 0.254 | 11.83 | 0.302 | 0.234 | 12.82 | 0.296 | 0.257 | 11.67 |
| 0.382 | 0.385 | 7.79 | 0.399 | 0.227 | 13.22 | 0.389 | 0.202 | 14.85 | 0.423 | 0.16 | 18.75 | 0.399 | 0.182 | 16.48 | 0.392 | 0.202 | 14.85 |
| 0.493 | 0.283 | 10.60 | 0.498 | 0.153 | 19.61 | 0.491 | 0.137 | 21.90 | 0.514 | 0.14 | 21.43 | 0.497 | 0.164 | 18.29 | 0.487 | 0.181 | 16.57 |
| 0.597 | 0.172 | 17.44 | 0.605 | 0.119 | 25.21 | 0.617 | 0.103 | 29.13 | 0.595 | 0.118 | 25.42 | 0.609 | 0.125 | 24.00 | 0.595 | [symbol] | 23.43 |
| 0.694 | 0.126 | 23.81 | 0.687 | 0.096 | 31.25 | 0.711 | 0.078 | 38.46 | 0.682 | 0.094 | 31.91 | 0.698 | 0.098 | 30.61 | 0.689 | 0.112 | 26.79 |
| 0.812 | 0.096 | 31.23 | 0.757 | 0.068 | 44.12 | 0.807 | 0.057 | 52.63 | 0.82 | 0.066 | 45.45 | 0.803 | 0.08 | 37.50 | 0.792 | 0.09 | 33.33 |
| 0.889 | 0.044 | 68.18 | 0.885 | 0.042 | 71.43 | 0.906 | 0.044 | 68.38 | 0.903 | 0.053 | 56.60 | 0.923 | 0.063 | 47.62 | 0.909 | 0.071 | 42.25 |

[symbol]

indicates data missing or illegible when filed

TABLE 12
Comparison between sensitivity (Sens) and positive predictive value (PPV) of the SVM model with 6 features plus Sybil score
on the NLST cohort. NNT = Number Needed to Treat to prevent one lung cancer based on number of patients based on positive
predictive value treated with an anti IL1 signaling agent with a hazard ratio similar to canakinumab hazard ratio of 0.3.

| Year 1 | | | Year 2 | | | Year 3 | | | Year 4 | | | Year 5 | | | Year 6 | | |
|---|---|---|---|---|---|---|---|---|---|---|---|---|---|---|---|---|---|
| Sens | PPV | NNT | Sens | PPV | NNT | Sens | PPV | NNT | Sens | PPV | NNT | Sens | PPV | NNT | Sens | PPV | NNT |
| 0.097 | 0.824 | 3.64 | 0.103 | 0.694 | 4.32 | 0.099 | 0.596 | 5.03 | 0.099 | 0.5 | 6.00 | 0.097 | 0.473 | 6.34 | 0.101 | 0.517 | 5.80 |
| 0.201 | 0.58 | 5.37 | 0.202 | 0.395 | 7.59 | 0.202 | 0.423 | 7.09 | 0.2 | 0.376 | 7.98 | 0.201 | 0.386 | 7.77 | 0.2 | 0.43 | 6.98 |
| [symbol] | 0.406 | 7.39 | 0.3 | 0.356 | 8.43 | 0.301 | 0.294 | 10.20 | 0.3 | 0.278 | 10.79 | 0.3 | 0.292 | 10.27 | [symbol] | 0.324 | 9.26 |
| 0.41 | 0.376 | 7.98 | 0.399 | 0.228 | 13.16 | 0.401 | 0.188 | 15.96 | 0.401 | 0.184 | 16.30 | 0.4 | 0.179 | 16.76 | 0.4 | 0.201 | 14.93 |
| 0.5 | 0.252 | 11.90 | 0.498 | 0.157 | 19.11 | 0.503 | 0.146 | [symbol] | 0.5 | 0.334 | 22.39 | 0.499 | 0.144 | 20.83 | 0.499 | 0.162 | 18.52 |
| 0.597 | 0.175 | 17.14 | 0.601 | 0.127 | 23.62 | 0.599 | 0.113 | 26.55 | 0.599 | 0.306 | 28.30 | 0.601 | 0.121 | 24.79 | 0.6 | 0.132 | 22.73 |
| 0.701 | 0.122 | 24.50 | 0.695 | 0.087 | 34.48 | 0.699 | 0.08 | 37.50 | 0.7 | 0.088 | 34.09 | 0.7 | 0.104 | 28.85 | [symbol] | 0.119 | 25.21 |
| 0.799 | 0.096 | 31.25 | 0.798 | 0.064 | 46.88 | 0.801 | 0.059 | 50.85 | 0.8 | 0.075 | 40.00 | 0.801 | 0.092 | 32.61 | 0.8 | 0.106 | 28.30 |
| 0.903 | 0.048 | 52.50 | 0.901 | 0.036 | 83.33 | 0.901 | 0.046 | 65.22 | 0.903 | 0.056 | 53.57 | 0.899 | 0.074 | 40.54 | 0.899 | 0.088 | 34.09 |

[symbol]

indicates data missing or illegible when filed

TABLE 13
Comparison between sensitivity (Sens) and positive predictive value (PPV) of the SVM model with 7 features plus Sybil score
on the NLST cohort. NNT = Number Needed to Treat to prevent one lung cancer based on number of patients based on positive
predictive value treated with an anti IL1 signaling agent with a hazard ratio similar to canakinumab hazard ratio of 0.3.

| Year 1 | | | Year 2 | | | Year 3 | | | Year 4 | | | Year 5 | | | Year 6 | | |
|---|---|---|---|---|---|---|---|---|---|---|---|---|---|---|---|---|---|
| Sens | PPV | NNT | Sens | PPV | NNT | Sens | PPV | NNT | Sens | PPV | NNT | Sens | PPV | NNT | Sens | PPV | NNT |
| 0.097 | 0.824 | 3.64 | 0.103 | 0.894 | 4.32 | 0.099 | 0.586 | 5.32 | 0.097 | 0.478 | 6.28 | 0.101 | 0.491 | 6.11 | 0.999 | 0.492 | 6.10 |
| 6.201 | 0.58 | 5.17 | 0.202 | [symbol] | 7.35 | [symbol] | 0.425 | 7.09 | 0.2 | [symbol] | 7.79 | 0.201 | 0.378 | 7.94 | 0.2 | 0.428 | 7.01 |
| 0.299 | 0.406 | 7.39 | 0.3 | 0.356 | 8.41 | [symbol] | 0.29 | 10.34 | 0.3 | 0.277 | 10.83 | 0.302 | [symbol] | [symbol] | 0.299 | 0.318 | 9.43 |
| 0.41 | 0.383 | 7.83 | 0.399 | 0.231 | [symbol] | [symbol] | 0.19 | 15.76 | 0.401 | 0.176 | 17.05 | 0.4 | [symbol] | [symbol] | 0.4 | 0.207 | 14.49 |
| 0.5 | 6.252 | [symbol] | 0.498 | 0.159 | 18.87 | 0.5 | [symbol] | [symbol] | 0.5 | 0.33 | 23.08 | 0.499 | 0.146 | 20.35 | [symbol] | [symbol] | 18.07 |
| 0.597 | 0.175 | 17.14 | [symbol] | 0.127 | 23.62 | 0.596 | 0.112 | 26.39 | 0.509 | 0.108 | 23.78 | 0.6 | 0.117 | 25.64 | 0.6 | [symbol] | 23.39 |
| 0.701 | 0.121 | [symbol] | 0.7 | 0.093 | 32.26 | [symbol] | [symbol] | 36.14 | 0.7 | 0.089 | 35.71 | 0.7 | 0.109 | 27.52 | 0.099 | [symbol] | 24.70 |
| [symbol] | 0.097 | 30.93 | 0.798 | 0.058 | 31.72 | 0.861 | [symbol] | 50.85 | 0.8 | 0.077 | 38.96 | 0.801 | 0.095 | 31.58 | 0.8 | [symbol] | 28.37 |
| 0.903 | [symbol] | 61.22 | 0.901 | 0.035 | 85.71 | 0.808 | [symbol] | 60.67 | 0.901 | 0.056 | 53.57 | 0.901 | 0.074 | 40.54 | 0.897 | [symbol] | 34.09 |

[symbol]

indicates data missing or illegible when filed

Overall, demonstrated is the feasibility and utility of a multimodal AI model capable of providing personalized cancer risk and use of anti IL-1 and anti-inflammatory therapeutics for pharmacoprophylaxis to improve the efficacy of lung cancer prevention via improved patient selection for benefit aka precision treatment. The artificial intelligence model has improved accuracy as compared to existing models based on clinical features alone. The model improves upon imaging based risk inference in the NL ST, and has validity in a diverse patient population in an urban academic medical center. Implementation of this model into practice requires further tuning and validation for generalizability in a prospective interventional trial. Such a trial would aim to no only evaluate model predictive performance, but additional meaningful outcomes of increased lung cancer screening participation and adherence among minorities which in turn would increase health equity.

The following paragraphs enumerated consecutively from 1 through 24 provide for various additional aspects of the present invention. In one embodiment, in a first paragraph:

1. A method to decrease bias in identifying the risk of cancer by using a multi modal artificial intelligence model and minimizing false positives to increase benefit of administering pharmacoprevention, the method comprising:
i) receiving, by an analysis device, one or more instances of radiographic image or images of an at risk organ for a subject;
ii) inputting, by the analysis device, a radiographic image or images of a patient to a classification neural network;
iii) identifying by recursive feature elimination the clinically significant features and subdividing the data into patients who have clinically significant features and patients who do not;
iv) inputting the subject's clinical features to the classification neural network; and
v) predicting, by the analysis device, a probability of developing cancer for the subject, wherein the classification neural network is trained using a cohort of radiographic images and/or clinical features.
whereby bias is decreased.

2. The method of paragraph 1, wherein the cancer is lung cancer, breast cancer, colon cancer, abdominal cancer, liver cancer, kidney cancer, brain cancer, head and neck cancer, prostate cancer and pelvic cancer.

3. The method of any of paragraphs 1-2, further comprising inputting the corresponding radiographic imaging of involved organs and clinical features.

4. The method of any of paragraphs 1-3, wherein the radiographic image or images comprise computed tomography images or magnetic resonance imaging.

5. The method of any of paragraphs 1-4, wherein the neural network comprises a residual neural network, first classification network, a convolutional neural network a diffusion neural network, a multi-modal neural network, a radial basis function neural network, a recurrent neural network, a generative adversarial neural network, a vision

6. The method of any of paragraphs 1-5 wherein the clinical features comprise one or more of: history of cancer, genetics, plasma, serum, blood, urine or sputum concentrations of C-Reactive Protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting, mammogram results and social determinant factors.

7. The method of any of paragraphs 1-6, wherein predicting comprises generating a numerical risk factor between 0 and 1, where a risk factor greater than about 0.015 is predictive of developing lung cancer within 1-10 years.

8. A method of treating a subject at enhanced risk of developing lung cancer comprising:
a. determining whether the subject is at enhanced risk of developing cancer using the artificial intelligence model of any of paragraphs 1-6; and
b. administering to the subject an effective pharmacopreventive prophylactic amount of an IL-1 signaling pathway antagonist.

9. The method of any of paragraphs 8, wherein the IL-1 signaling pathway antagonist comprises: anakinra, MCC950 (CP-456773), CY-09, Oridonin, Tranilast, MNS, OLT1177 dapansutrile, Bay 11-7082, BOT-4-one, Parthenolide, and INF39, rilonacept, VX 765, ILIRAP, nadulonimab, a JAK inhibitor.

10. The method of any of paragraphs 1-9, wherein the IL-1 signaling pathway antagonist comprises an antibody to human IL-1b.

11. The method of any of paragraphs 1-10, wherein the antibody comprises canakinumab.

12. The method of any of paragraphs 1-11, wherein the antibody comprises a sweeping antibody.

13. The method of any of paragraphs 1-12, wherein the IL-1 signaling antagonist comprises an inhibitor to the IL-1 receptor.

14. The method of any of paragraphs 1-13, wherein the IL-1 inhibitor comprises anakinra.

15. The method of any of paragraphs 1-14, wherein the IL-1 signaling pathway antagonist comprises a JAK inhibitor.

16. The method of any of paragraphs 1-15, wherein the JAK inhibitor comprises ruxolitinib, tofacitinib, oclacitinib, baricitinib, peficitinib, upadacitinib, fedratinib, delgocitinib, filgotinib, abrocitinib, pacritinib, deucravacitinib, ritlecitinib, or momelotinib.

17. The method of any of paragraphs 1-16, wherein the cancer is lung cancer, breast cancer, colon cancer, abdominal cancer, liver cancer, kidney cancer, brain cancer, prostate cancer, head and neck cancer and pelvic cancer.

18. A method for determining the efficacy of a cancer treatment or treating agent using a multi modal artificial intelligence model, the method comprising:
a. administering the treatment or treating agent to a test subject;
b. receiving, by an analysis device, one or more instances of radiographic image or images of the lungs for a subject;
c. inputting, to the analysis device, the radiographic image or images of the lungs to a first classification neural network;
d. optionally inputting the subject's clinical features comprising history of cancer, genetics, plasma, serum, blood, urine or sputum concentrations of C-reactive protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting and social determinant factors of health; and
e. assigning, by the analysis device, a numerical value indicative of the efficacy of the treatment or treating agent, wherein the first classification neural network is trained using a cohort of radiographic images of the lungs and/or clinical features.

19. The method of any of paragraphs 1-18, wherein the cancer comprises lung cancer, breast cancer, colon cancer, abdominal cancer, liver cancer, kidney cancer, brain cancer, prostate cancer, head and neck cancer and pelvic cancer, head and neck cancer.

20. The method of any of paragraphs 1-19, wherein the cancer is breast cancer, wherein:
the input features comprise mammograms and breast MRI; and the clinical features comprise age, family history of breast cancer, age of menarche, age of menopause, number of pregnancies, BMI, smoking history, history of estrogen containing medicines, history of hormone replacement therapy, history of breast or chest radiation.

21. The method of any of paragraphs 1-20, wherein pharmacoprevention for breast cancer comprises:
a. determining whether the subject is at enhanced risk of developing breast cancer using the artificial intelligence model of any of paragraphs 1-20; and
b. administering to the subject an effective prophylactic amount of an estrogen receptor modulator or antagonist comprising tamoxifen, toremifene, raloxifene, bazedoxifene, anastrozole, letrozole, exemestane, fulvestrant, elacestrant, giredestrant, camizestrant, lasofoxifene, rintodestrant, imlunestrant, op-1250, lsz102; and/or
c. administering to the subject an effective prophylactic amount of a GLP-1 analog comprising semaglutide, tirzepatide; and/or
d. administering to the subject an effective prophylactic amount of a GLP receptor agonist comprising orforglipron;
wherein the progress of the cancer is monitored by the artificial intelligence model of paragraph 1.

22. The method of any of paragraphs 1-21, wherein the cancer is prostate cancer, wherein:
the input features comprise prostate-specific antigen test, rectal exam, urination problems or biopsy and MRI scan; and
the clinical features comprise age, family history of prostate cancer and, obesity.

23 The method of any of paragraphs 1-22, wherein the pharmacoprevention comprises:
a. determining whether the subject is at enhanced risk of developing breast cancer using the artificial intelligence model of paragraph 1; and
b. administering to the subject an effective prophylactic amount of an radiation therapy or radiopharmaceutical therapy, hormone therapy, chemotherapy, immunotherapy, bisphosphonate therapy;
wherein the progress of the cancer is monitored by the artificial intelligence model of paragraphs 1-22.

24. A method that reduces false positives in cancer prediction by incorporation of medical imaging into a multimodal AI model to improve precision comprising:
incorporating a computer vision inference of medical imaging performed for cancer screening;
incorporating into the computer vision model a multimodal AI framework a support vector machine model or decision tree model;
determining a positive predictive value or the likelihood that a patient predictive value is positive;
wherein the method incorporates the computer vision inference from one medical imaging study, or a series of medical imaging studies to improve sensitivity and specificity.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

### REFERENCES

The following references are presented solely as background information.
1. Wloszek, E., Krupa, K., Skrok, E., Budzik, M. P., Deptala, A., and Badowska-Kozakiewicz, A. (2025). HPV and Cervical Cancer-Biology, Prevention, and Treatment Updates. Curr Oncol 32. 10.3390/curroncol32030122.
2. Fisher, B., Costantino, J. P., Wickerham, D. L., Redmond, C. K., Kavanah, M., Cronin, W. M., Vogel, V., Robidoux, A., Dimitrov, N., Atkins, J., et al. (1998). Tamoxifen for prevention of breast cancer: report of the National Surgical Adjuvant Breast and Bowel Project P-1 Study. Journal of the National Cancer Institute 90, 1371-1388. 10.1093/jnci/90.18.1371.
3. Vogel, V. G., Costantino, J. P., Wickerham, D. L., Cronin, W. M., Cecchini, R. S., Atkins, J. N., Bevers, T. B., Fehrenbacher, L., Pajon, E. R., Jr., Wade, J. L., 3rd, et al. (2006). Effects of tamoxifen vs raloxifene on the risk of developing invasive breast cancer and other disease outcomes: the NSABP Study of Tamoxifen and Raloxifene (STAR) P-2 trial. JAMA 295, 2727-2741. 10.1001/jama.295.23.joc60074.
4. Thorneloe, R. J., Hall, L. H., Walter, F. M., Side, L., Lloyd, K. E., Smith, S. G., and Investigators, E. (2020). Knowledge of Potential Harms and Benefits of Tamoxifen among Women Considering Breast Cancer Preventive Therapy. Cancer prevention research 13, 411-422. 10.1158/1940-6207.CAPR-19-0424.
5. Omenn, G. S., Goodman, G. E., Thornquist, M. D., Balmes, J., Cullen, M. R., Glass, A., Keogh, J. P., Meyskens, F. L., Valanis, B., Williams, J. H., et al. (1996). Effects of a combination of beta carotene and vitamin A on lung cancer and cardiovascular disease. N Engl J Med 334, 1150-1155. 10.1056/NEJM199605023341802.
6. Soneji, S., Tanner, N. T., Silvestri, G. A., Lathan, C. S., and Black, W. (2017). Racial and Ethnic Disparities in Early-Stage Lung Cancer Survival. Chest 152, 587-597. 10.1016/j.chest.2017.03.059.
7. Watson, K. S., Hulbert, A., Henderson, V., Chukwudozie, I. B., Aponte-Soto, L., Lerner, L., Martinez, E., Kim, S., and Winn, R. A. (2019). Lung Cancer Screening and Epigenetics in African Americans: The Role of the Socioecological Framework. Front Oncol 9, 87. 10.3389/fonc.2019.00087.
8. National Lung Screening Trial Research, T., Aberle, D. R., Adams, A. M., Berg, C. D., Black, W. C., Clapp, J. D., Fagerstrom, R. M., Gareen, I. F., Gatsonis, C., Marcus, P. M., and Sicks, J. D. (2011). Reduced lung-cancer mortality with low-dose computed tomographic screening. N Engl J Med 365, 395-409. 10.1056/NEJMoa1102873.
9. Pinsky, P. F., Ford, M., Gamito, E., Higgins, D., Jenkins, V., Lamerato, L., Tenorio, S., Marcus, P. M., and Gohagan, J. K. (2008). Enrollment of racial and ethnic minorities in the Prostate, Lung, Colorectal and Ovarian Cancer Screening Trial. J Natl Med Assoc 100, 291-298. 10.1016/s0027-9684(15)31241-4.
10. Pinsky, P. F., Lau, Y. K., and Doubeni, C. A. (2021). Potential Disparities by Sex and Race or Ethnicity in Lung Cancer Screening Eligibility Rates. Chest 160, 341-350. 10.1016/j.chest.2021.01.070.
11. Choi, E., Ding, V. Y., Luo, S. J., Ten Haaf, K., Wu, J. T., Aredo, J. V., Wilkens, L. R., Freedman, N. D., Backhus, L. M., Leung, A. N., et al. (2023). Risk Model-Based Lung Cancer Screening and Racial and Ethnic Disparities in the US. JAMA Oncol. 10.1001/jamaoncol.2023.4447.
12. Siegel, R. L., Miller, K. D., Fuchs, H. E., and Jemal, A. (2022). Cancer statistics, 2022. CA Cancer J Clin 72, 7-33. 10.3322/caac.21708.
13. Byrne, C. A., Gomez, S. L., Kim, S., Oddo, V. M., Koh, T. J., and Fantuzzi, G. (2022). Disparities in inflammation between non-Hispanic black and white individuals with lung cancer in the Greater Chicago Metropolitan area. Front Immunol 13, 1008674. 10.3389/fimmu.2022.1008674.
14. Kim, S. J., Kery, C., An, J., Rineer, J., Bobashev, G., and Matthews, A. K. (2024). Racial/Ethnic disparities in exposure to neighborhood violence and lung cancer risk in Chicago. Soc Sci Med 340, 116448. 10.1016/j.socscimed.2023.116448.
15. Mahmoud, A. M., Brister, E., David, O., Valyi-Nagy, K., Sverdlov, M., Gann, P. H., and Kim, S. J. (2023). Machine Learning for Digital Scoring of PRMT6 in Immunohistochemical Labeled Lung Cancer. Cancers (Basel) 15. 10.3390/cancers15184582.
16. (2020). In Smoking Cessation: A Report of the Surgeon General.
17. Tyrer, J., Duffy, S. W., and Cuzick, J. (2004). A breast cancer prediction model incorporating familial and personal risk factors. Stat Med 23, 1111-1130. 10.1002/sim.1668.
18. Gail, M. H., Brinton, L. A., Byar, D. P., Corle, D. K., Green, S. B., Schairer, C., and Mulvihill, J. J. (1989). Projecting individualized probabilities of developing breast cancer for white females who are being examined annually. Journal of the National Cancer Institute 81, 1879-1886. 10.1093/jnci/81.24.1879.
19. Tammemagi, M. C., Katki, H. A., Hocking, W. G., Church, T. R., Caporaso, N., Kvale, P. A., Chaturvedi, A. K., Silvestri, G. A., Riley, T. L., Commins, J., and Berg, C. D. (2013). Selection criteria for lung-cancer screening. N Engl J Med 368, 728-736. 10.1056/NEJMoa1211776.
20. Bach, P. B., Kattan, M. W., Thornquist, M. D., Kris, M. G., Tate, R. C., Barnett, M. J., Hsieh, L. J., and Begg, C. B. (2003). Variations in lung cancer risk among smokers. Journal of the National Cancer Institute 95, 470-478. 10.1093/jnci/95.6.470.
21. Ridker, P. M., Everett, B. M., Thuren, T., MacFadyen, J. G., Chang, W. H., Ballantyne, C., Fonseca, F., Nicolau, J., Koenig, W., Anker, S. D., et al. (2017). Antiinflammatory Therapy with Canakinumab for Atherosclerotic Disease. N Engl J Med 377, 1119-1131. 10.1056/NEJMoa1707914.
22. Hill, W., Lim, E. L., Weeden, C. E., Lee, C., Augustine, M., Chen, K., Kuan, F. C., Marongiu, F., Evans, E. J., Jr., Moore, D. A., et al. (2023). Lung adenocarcinoma promotion by air pollutants. Nature 616, 159-167. 10.1038/s41586-023-05874-3.
23. Ahn, G., Banik, S. M., and Bertozzi, C. R. (2021). Degradation from the outside in: Targeting extracellular and membrane proteins for degradation through the endolysosomal pathway. Cell Chem Biol 28, 1072-1080. 10.1016/j.chembiol.2021.02.024.
24. Pedregosa, F., Varoquaux, G., Gramfort, A., Michel, V., Thirion, B., Grisel, O., Blondel, M., Prettenhofer, P., Weiss, R., and Dubourg, V. (2011). Scikit-learn: Machine learning in Python. the Journal of machine Learning research 12, 2825-2830.
25. Mikhael, P. G., Wohlwend, J., Yala, A., Karstens, L., Xiang, J., Takigami, A. K., Bourgouin, P. P., Chan, P., Mrah, S., Amayri, W., et al. (2023). Sybil: A Validated Deep Learning Model to Predict Future Lung Cancer Risk From a Single Low-Dose Chest Computed Tomography. J Clin Oncol 41, 2191-2200. 10.1200/JCO.22.01345.
26. DeLong, E. R., DeLong, D. M., and Clarke-Pearson, D. L. (1988). Comparing the areas under two or more correlated receiver operating characteristic curves: a nonparametric approach. Biometrics 44, 837-845.
27. Demler, O. V., Pencina, M. J., and D'Agostino, R. B., Sr. (2012). Misuse of DeLong test to compare AUCs for nested models. Stat Med 31, 2577-2587. 10.1002/sim.5328.
28. Hanley, J. A., and McNeil, B. J. (1982). The meaning and use of the area under a receiver operating characteristic (ROC) curve. Radiology 143, 29-36. 10.1148/radiology.143.1.7063747.
29. Boyd, A. D., Gonzalez-Guarda, R., Lawrence, K., Patil, C. L., Ezenwa, M. O., O'Brien, E. C., Paek, H., Braciszewski, J. M., Adeyemi, O., Cuthel, A. M., et al. (2023). Equity and bias in electronic health records data. Contemp Clin Trials 130, 107238. 10.1016/j.cct.2023.107238.
30. Boyd, A. D., Gonzalez-Guarda, R., Lawrence, K., Patil, C. L., Ezenwa, M. O., O'Brien, E. C., Paek, H., Braciszewski, J. M., Adeyemi, O., Cuthel, A. M., et al. (2023). Potential bias and lack of generalizability in electronic health record data: reflections on health equity from the National Institutes of Health Pragmatic Trials Collaboratory. J Am Med Inform Assoc 30, 1561-1566. 10.1093/jamia/ocad115.
31. Crowe, B., and Rodriguez, J. A. (2024). Identifying and Addressing Bias in Artificial Intelligence. JAMA Netw Open 7, e2425955. 10.1001/jamanetworkopen.2024.25955.
32. Ratwani, R. M., Sutton, K., and Galarraga, J. E. (2024). Addressing AI Algorithmic Bias in Health Care. JAMA. 10.1001/jama.2024.13486.
33. Vishwanatha, J. K., Christian, A., Sambamoorthi, U., Thompson, E. L., Stinson, K., and Syed, T. A. (2023). Community perspectives on AI/ML and health equity: AIM-AHEAD nationwide stakeholder listening sessions. PLOS Digit Health 2, e0000288. 10.1371/journal.pdig.0000288.
34. Cancer Data and Image Database for AI (CANDID-4-AI).
35. Carter-Harris, L., and Gould, M. K. (2017). Multilevel Barriers to the Successful Implementation of Lung Cancer Screening: Why Does It Have to Be So Hard?Ann Am Thorac Soc 14, 1261-1265. 10.1513/AnnalsATS.201703-204PS.
36. Lopez-Olivo, M. A., Maki, K. G., Choi, N. J., Hoffman, R. M., Shih, Y. T., Lowenstein, L. M., Hicklen, R. S., and Volk, R. J. (2020). Patient Adherence to Screening for Lung Cancer in the US: A Systematic Review and Meta-analysis. JAMA Netw Open 3, e2025102. 10.1001/jamanetworkopen.2020.25102.

1. A method to decrease bias in identifying the risk of cancer by using a multi modal artificial intelligence model and minimizing false positives to increase benefit of administering pharmacoprevention, the method comprising:
i) receiving, by an analysis device, one or more instances of radiographic image or images of an at risk organ for a subject;
ii) inputting, by the analysis device, a radiographic image or images of a patient to a classification neural network;
iii) identifying by recursive feature elimination the clinically significant features and subdividing the data into patients who have clinically significant features and patients who do not;
iv) inputting the subject's clinical features to the classification neural network; and
v) predicting, by the analysis device, a probability of developing cancer for the subject, wherein the classification neural network is trained using a cohort of radiographic images and/or clinical features;
whereby bias is decreased.

2. The method of claim 1, wherein the cancer is lung cancer, breast cancer, colon cancer, abdominal cancer, liver cancer, kidney cancer, brain cancer, head and neck cancer, prostate cancer and pelvic cancer.

3. The method of claim 2, further comprising inputting the corresponding radiographic imaging of involved organs and clinical features.

4. The method of claim 1, wherein the radiographic image or images comprise computed tomography images or magnetic resonance imaging.

5. The method of claim 1, wherein the neural network comprises a residual neural network, first classification network, a convolutional neural network a diffusion neural network, a multi-modal neural network, a radial basis function neural network, a recurrent neural network, a generative adversarial neural network, a vision

6. The method of claim 1 wherein the clinical features comprise one or more of: history of cancer, genetics, plasma, serum, blood, urine or sputum concentrations of C-Reactive Protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting, mammogram results and social determinant factors.

7. A method of treating a subject at enhanced risk of developing lung cancer comprising
a. determining whether the subject is at enhanced risk of developing cancer using the artificial intelligence model of claim 1; and
b. administering to the subject an effective pharmacopreventive prophylactic amount of an IL-1 signaling pathway antagonist.

8. The method of claim 7, wherein the IL-1 signaling pathway antagonist comprises: anakinra, MCC950 (CP-456773), CY-09, Oridonin, Tranilast, MNS, OLT1177 dapansutrile, Bay 11-7082, BOT-4-one, Parthenolide, and INF39, rilonacept, VX 765, ILIRAP, nadulonimab, a JAK inhibitor.

9. The method of claim 7, wherein the IL-1 signaling pathway antagonist comprises an antibody to human IL-1b.

10. The method of claim 9, wherein the antibody comprises canakinumab.

11. The method of claim 9, wherein the antibody comprises a sweeping antibody

12. The method of claim 7, wherein the IL-1 signaling antagonist comprises an inhibitor to the IL-1 receptor.

13. The method of claim 12, wherein the IL-1 inhibitor comprises anakinra.

14. The method of claim 12, wherein the IL-1 signaling pathway antagonist comprises a JAK inhibitor.

15. The method of claim 14, wherein the JAK inhibitor comprises ruxolitinib, tofacitinib, oclacitinib, baricitinib, peficitinib, upadacitinib, fedratinib, delgocitinib, filgotinib, abrocitinib, pacritinib, deucravacitinib, ritlecitinib, or momelotinib.

16. A method for determining the efficacy of a cancer treatment or treating agent using a multi modal artificial intelligence model, the method comprising:
a. administering the treatment or treating agent to a test subject;
b. receiving, by an analysis device, one or more instances of radiographic image or images of the lungs for a subject;
c. inputting, to the analysis device, the radiographic image or images of the cancer to a first classification neural network;
d. optionally inputting the subject's clinical features comprising history of cancer, genetics, plasma, serum, blood, urine or sputum concentrations of C-reactive protein, carcinogen embryonic antigen, cell-free DNA or RNA and chemical modifications thereof, PSA levels, rectal exam, residential history, environmental exposures, age, race, education, BMI, presence of COPD, personal history of cancer, family history of cancer, smoking status, cigarettes per day, duration of smoking, duration of quitting and social determinant factors of health; and
e. assigning, by the analysis device, a numerical value risk factor indicative of the efficacy of the treatment or treating agent, wherein the first classification neural network is trained using a cohort of radiographic images of the lungs and/or clinical features.

17. The method of claim 16, wherein the cancer comprises lung cancer, breast cancer, colon cancer, abdominal cancer, liver cancer, kidney cancer, brain cancer, prostate cancer, head and neck cancer and pelvic cancer, head and neck cancer.

18. The method of claim 17, wherein the cancer is breast cancer, wherein:
the input features comprise mammograms and breast MRI; and
the clinical features comprise age, family history of breast cancer, age of menarche, age of menopause, number of pregnancies, BMI, smoking history, history of estrogen containing medicines, history of hormone replacement therapy, history of breast or chest radiation.

19. The method of claim 18, wherein pharmacoprevention for breast cancer comprises
a. determining whether the subject is at enhanced risk of developing breast cancer using the artificial intelligence model of claim 1; and
b. administering to the subject an effective prophylactic amount of an estrogen receptor modulator or antagonist comprising tamoxifen, raloxifene, anastrozole, letrozoland, fulvestrant; and/or
c. administering to the subject an effective prophylactic amount of a GLP-1 analog comprising semaglutize, tirzepatide; and/or
d. administering to the subject an effective prophylactic amount of a GLP receptor agonist comprising orforglipron;
wherein the progress of the cancer is monitored by the artificial intelligence model of claim 1.

20. The method of claim 17, wherein the cancer is prostate cancer, wherein:
the input features comprise prostate-specific antigen test, rectal exam, urination problems or biopsy and PET/PSMA PET or MRI scan; and
the clinical features comprise age, family history of prostate cancer and, obesity.

21. The method of claim 20, wherein the pharmacoprevention comprises:
a. determining whether the subject is at enhanced risk of developing breast cancer using the artificial intelligence model of claim 1; and
b. administering to the subject an effective prophylactic amount of a radiation therapy or radiopharmaceutical therapy, anti androgen inhibitors comprising abiraterone acetate, enzalutamide, apalutamide, darolutamide, leuprolide, goserelin, triptorelin, histrelin, degarelix, relugolix, bicalutamide, flutamide, nilutamide, finasteride and dutasteride;
wherein the progress of the cancer is monitored by the artificial intelligence model of claim 1.
